(19) United States
(12) Patent Application Publication
(10) Pub. No.: US 2005/0137247 A1
(43) Pub. Date: Jun. 23, 2005

(54) Methods and compositions for treatment of hypertension

(75) Inventors: Charles A. Czeisler, Sherborn, MA (US); Frank A.J.L. Scheer, Brighton, MA (US)

Correspondence Address: FOLEY HOAG, LLP;PATENT GROUP, WORLD TRADE CENTER WEST, 155 SEAPORT BLVD, BOSTON, MA, 02110, US

(73) Assignee: The Brigham and Women's Hospital, Inc., Boston, MA (US)

(21) Appl. No.: 11/020,626

(22) Filed: Dec. 22, 2004

Related U.S. Application Data
(60) Provisional application No. 60/531,769, filed on Dec. 22, 2003.

(51) Int. Cl. A61K 31/724; A61K 31/405; A61K 31/381; A61K 31/343

(52) U.S. Cl. 514/419; 514/469; 514/58; 514/443

(57) ABSTRACT

Methods and compositions for treating and/or preventing hypertension are provided. The methods involve administration of melatonin, or an analog thereof, to a subject. The methods and compositions may be used to treat various forms of hypertension, including essential hypertension.

### RELATED APPLICATIONS

This application claims the benefit of U.S. Provisional Patent Application Ser. No. 60/531,769, filed Dec. 22, 2003, incorporated herein in its entirety by this reference. The entire contents of PCT Application Ser. No. PCT/US2004/______, entitled “Methods and Compositions for Treatment of Hypertension,” having Foley Hoag LLP Docket Number BWY-013.25 and filed on Dec. 22, 2004, is hereby incorporated herein by this reference.

### BACKGROUND

Hypertension is defined as abnormally elevated blood pressure. More specifically, when a person under conditions of rest consistently has a blood pressure that exceeds 140/90 (systole/diastole), the person is said to have high blood pressure or hypertension. It is currently believed that over fifty million people in the United States have hypertension and fifteen to twenty percent of all deaths in people over fifty years of age occur as a direct or indirect result of hypertension. Actuarial statistics show that the disability and mortality rates of hypertensive persons are higher for each age bracket than for persons with normal blood pressure. Specific ailments attributable to hypertension include heart failure, myocardial infarction, rupture or thrombus of the blood vessels in the brain and kidney damage.

Current treatments for hypertension include, for example, beta-blockers, alpha-blockers, angiotensin-converting enzyme (ACE) inhibitors, diuretics, angiotensin II inhibitors, and Ca²⁺ antagonists. Unfortunately, in some cases these treatments may be inadequate do to insufficient therapeutic effects or the severity of unwanted side effects. For example, diruetic based anithypertensives may lead to hypocalcemia, hyperuricemia, glycosemia, hyperlipidemia, etc., vasodilating antihypertensives may induce tachycardia, arrhythmia, headache, etc., angiotensin II inhibitors have been associated with decreased renal function, uremia, heart failure, etc., and Ca²⁺ antagonists may inhibit cardiac function due to reduced myocardial contraction and induce disorders involving insulin secretion. Accordingly, a need still exists for safe and effective antihypertensive agents which can produce therapeutically effective reductions in blood pressure in the absence of unwanted or dangerous side effects.

### SUMMARY

Provided herein are methods and compositions for treating hypertension.

In one aspect, a method for treating hypertension is provided, wherein the method comprises administering to a subject in need thereof a melatonin receptor agonist, wherein said melatonin receptor agonist is administered to said subject at night time on a daily basis, and wherein said melatonin receptor agonist reduces blood pressure in said subject thereby treating hypertension.

In certain embodiments, the melatonin receptor agonist may be administered at night time for at least 2, 7, 14, or 21, or more days.

In certain embodiments, the melatonin receptor agaonist is a compound of Formula I: 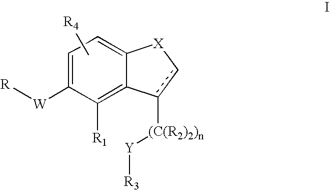
wherein, independently for each occurrence:
R is C₁₋₆ alkyl, C₂₋₈ alkenyl, C₂₋₈ alkynyl, cycloalkyl, aryl, aralkyl, heteroaryl, heteroaralkyl, —CO₂(C₁₋₆ alkyl), —CO₂(aryl), —C(O)NH(C₁₋₆ alkyl), —C(O)NH(aryl), —C(O)H, —C(O)(aryl), or —C(O)(C₁₋₆ alkyl);
R₁ is H, C₁₋₆ alkyl, C₂₋₈ alkenyl, C₂₋₈ alkynyl, aryl, aralkyl, heteroaryl, heteroaralkyl; or R and R₁ taken together form a fused aromatic, cycloalkyl, or cycloalkenyl ring;
R₂ is H, C₁₋₆ alkyl, cycloalkyl, aryl, aralkyl, or —CO₂R₁;
R₃ is H, C₁₋₆ alkyl, cycloalkyl, aryl, aralkyl, or —C(O)R₁;
R₄ is H, C₁₋₆ alkyl, or halide;
R₅ is H, C₁₋₆ alkyl, C₁₋₆ alkoxy; aryl, aralkyl, cycloalkyl, —N(R₁)₂, or heteroaryl;
W is O, N(R₂), or S;
X is CH₂, O, N(R₂) or S;
Y is O, N(R₆), or S;
R₆ is H, C₁₋₆ alkyl, —CO₂(C₁₋₆ alkyl); or R₆ and R₃ taken together form a ring;
the 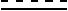
   line indicates either a single or double bond between the two carbon atoms; and
n is an integer from 1 to 6 inclusive;
or a pharmaceutically acceptable salt, solvate, clathrate, polymorph, or co-crystal thereof.

In certain embodiments, the melatonin receptor agonist is a compound of Formula II: 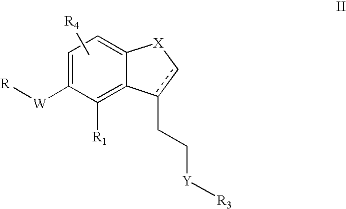
wherein, independently for each occurrence:
R is C₁₋₆ alkyl, C₂₋₈ alkenyl, C₂₋₈ alkynyl, cycloalkyl, aryl, aralkyl, heteroaryl, heteroaralkyl, —CO₂(C₁₋₆ alkyl), —CO₂(aryl), —C(O)NH(C₁₋₆ alkyl), —C(O)NH(aryl), —C(O)H, —C(O)(aryl), or —C(O)(C₁₋₆ alkyl);
R₁ is H, C₁₋₆ alkyl, C₂₋₈ alkenyl, C₂₋₈ alkynyl, aryl, aralkyl, heteroaryl, heteroaralkyl; or R and R₁ taken together form a fused aromatic, cycloalkyl, or cycloalkenyl ring;
R₃ is H, C₁₋₆ alkyl, cycloalkyl, aryl, aralkyl, or —C(O)R₁;
R₄ is H, C₁₋₆ alkyl, or halide;
W is O or S;
X is CH₂, O, N(R₁) or S;
Y is O, N(R₆), or S;
R₆ is H, C₁₋₆ alkyl, —CO₂(C₁₋₆ alkyl); or R₆ and R₃ taken together form a ring; and
the 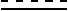
   line indicates either a single or double bond between the two carbon atoms;
or a pharmaceutically acceptable salt, solvate, clathrate, polymorph, or co-crystal thereof.

In certain embodiments, the melatonin receptor agonist is a compound of Formula III: 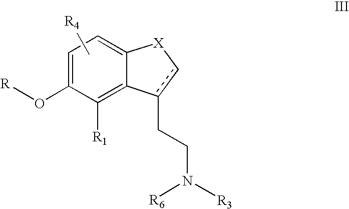
wherein, independently for each occurrence:
R is C₁₋₆ alkyl, C₂₋₈ alkenyl, C₂₋₈ alkynyl, cycloalkyl, aryl, aralkyl, heteroaryl, heteroaralkyl, —CO₂(C₁₋₆ alkyl), —CO₂(aryl), —C(O)NH(C₁₋₆ alkyl), —C(O)NH(aryl), —C(O)H, —C(O)(aryl), or —C(O)(C₁₋₆ alkyl);
R₁ is H, C₋₆ alkyl, C₂₋₈ alkenyl, C₂₋₈ alkynyl, aryl, aralkyl, heteroaryl, heteroaralkyl; or R and R₁ taken together form a fused aromatic, cycloalkyl, or cycloalkenyl ring;
R₃ is H, C₁₋₆ alkyl, cycloalkyl, aryl, aralkyl, or —C(O)R₁;
R₄ is H, C₁₋₆ alkyl, or halide;
X is CH₂ or N(R₁);
R₆ is H, C₁₋₆ alkyl, —CO₂(C₁₋₆ alkyl); or R₆ and R₃ taken together form a ring; and
the 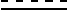
   line indicates either a single or double bond between the two carbon atoms;
or a pharmaceutically acceptable salt, solvate, clathrate, polymorph, or co-crystal thereof.

In certain embodiments, the melatonin receptor agonist is a compound of Formula IV: 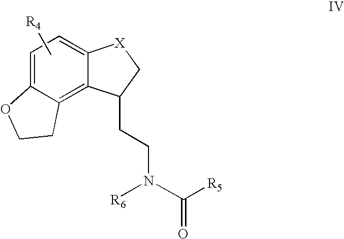
wherein, independently for each occurrence:
R₄ is H, C₁₋₆ alkyl, or halide;
R₅ is H, C₁₋₆ alkyl, C₁₋₆ alkoxy; aryl, aralkyl, cycloalkyl, or heteroaryl;
X is CH₂ or N(R₅); and
R₆ is H, C₁₋₆ alkyl, —CO₂(C₁₋₆ alkyl);
or a pharmaceutically acceptable salt, solvate, clathrate, polymorph, or co-crystal thereof.

In certain embodiments, the present invention relates to a method of treating hypertension in a patient comprising administering to the patient a melatonin receptor agonist wherein the melatonin receptor agonist is one or more of the following: N-[2-(5-Methoxy-1H-indol-3-yl)ethyl]acetamide (TAK-375), N-[2-(3-ethyl-7-methoxynaphthyl)ethyl]-acetamide (S21634), N-[2-(7-methoxynaphth-1-yl)-ethyl]-acetamide (S20098), N-[2-naphth-1-yl-ethyl]-cyclobutyl carboxamide (S20928), 2-iodomelatonin, N-acetyl-5-HT, LY 156735, BMS-214778, melatonin, agomelatine, CGP 52608, low-dose melatonin A, GR196429, S20242, S23478, S24268, S25150, melatonin receptor research compound A, GW290569, controlled release melatonin, luzindole, GR135531, melatonin agonist A, melatonin analogue B, melatonin agonist C, melatonin agonist D, melatonin agonist E, melatonin agonist F, melatonin agonist G, melatonin agonist H, melatonin agonist I, melatonin analog J, melatonin analog K, melatonin analog L, AH-001, GG-012, enol-3-IPA, ML-23, SL-18.1616, IP-100-9, melatonin low-dose B, sleep inducing peptide A, oros-melatonin, AH-017, AH-002, IP-101, 5-hydroxy-N-acetyl-tryptamine (NAT), 5-methoxy-N-bu-tanoyltryptamine (bMT), prazosin, phenylmelatonin, seradrene, β-methyl-6-chloromelatonin, 5-hydroxyethoxy-N-acetyltryptamine (5-HEAT), 8-methoxy-2-propionamidotetralin, PD-6735, seroctin, N-[2-(5-methoxy-2-phenylfuro[2,3-b]pyridin-3-yl)ethyl]acetamide, N-[2-(5-methoxy-2-phenylfuro[2,3-c]pyridin-3-yl)ethyl]acetamide, N-[(±)-2-(7-methoxy-1,2,3,4-tetrahydro-1-naphthyl)ethyl]cyclopropyl-carboxamide, N-[2-(7-methoxy-1-naphthyl)ethyl]acetamide, N-acetyl-4-aminomethyl-6-methoxy-9-methyl-1,2,3,4-tetrahydrocarbazole (AMMTC), 3-(2-aminopropyl)indole, 6-chloromelatonin, 2,3-dihydromelatonin, 6-chloro-2,3-dihydromelatonin, N-acetyl-N′-formyl-5-methoxykynurenamine, 6-methoxybenzoxazolinone, or a pharmaceutically acceptable salt, solvate, clathrate, polymorph, or co-crystal thereof.

In certain embodiments, the present invention relates to a method of treating hypertension in a patient comprising administering to the patient a melatonin receptor agonist wherein the melatonin receptor agonist is one or more of the following: N-[2-(5-Methoxy-1H-indol-3-yl)ethyl]acetamide (TAK-375), N-[2-(3-ethyl-7-methoxynaphthyl)ethyl]-acetamide (S21634), N-[2-(7-methoxynaphth-1-yl)-ethyl]-acetamide (S20098), N-[2-naphth-1-yl-ethyl]-cyclobutyl carboxamide (S20928), 2-iodomelatonin, N-acetyl-5-HT, LY 156735, BMS-214778, melatonin, or a pharmaceutically acceptable salt, solvate, clathrate, polymorph, or co-crystal thereof.

In certain embodiments, the methods and compositions described herein may be used to treat various forms of hypertension, including, for example, essential hypertension. In a certain embodiment, the methods and compositions described herein may be used to reduce systolic blood pressure by about 6 mm Hg and/or diastolic blood pressure by about 4 mm Hg. In certain embodiments, a reduction in blood pressure may be monitored by determining a subject'methoxy-N-bu-tanoyltryptamines ambulatory blood pressure.

In certain embodiments, about 0.05, 0.1, 0.3, 1, 2.5, 5, 10, 20, 30, 50, 64, 100, 200, or 300 mg of said melatonin receptor agonist is administered to said subject at night time on a daily basis.

In certain embodiment, the melatonin receptor agonist is administered to said subject about 1 hour before bedtime and may be formulated for oral, nasal, parenteral, or transdermal administration.

In certain embodiments, the methods and compositions described herein may be used to treat a human subject suffering from hypertension. In certain embodiments, administration of said melatonin receptor agonist does not affect the heart rate of said subject.

In certain embodiments, the methods described herein may also comprise identifying a subject suffering from hypertension and/or monitoring the blood pressure of said subject, including monitoring blood pressure on a regular basis, including a daily basis.

In another aspect, a method for treating hypertension is provided, wherein the method comprises:
a) administering to a subject a melatonin receptor agonist at night time;
b) obtaining a blood pressure reading for said subject; and
c) repeating steps a) and b) on a daily basis until a desirable level of blood pressure reduction is achieved in said subject, thereby treating hypertension.

In another aspect, a treatment program for subjects with hypertension is provided, wherein the treatment program comprises:
a) a supply of a melatonin receptor agonist sufficient for a daily night time dose of at least 1 mg per day; and
b) a blood pressure monitor for regular blood pressure monitoring;
wherein daily ingestion of said melatonin receptor agonist for at least 7 days reduces blood pressure in a subject.

In another aspect, a program for treating hypertension in a subject is provided, wherein the program comprises:
a) determining a first blood pressure reading for a subject with hypertension;
b) providing a supply of a melatonin receptor agonist;
c) directing said subject to ingest a dose of said melatonin receptor agonist on a regular basis at night time for a period;
d) determining a second blood pressure reading for said subject at about the end of the period; and
e) comparing said second blood pressure reading with said first blood pressure reading to determine any reduction in hypertension in said subject during said period.

In certain embodiments, the method may further comprise directing said subject to monitor said subject's blood pressure during said period.

In certain embodiments, the method may further comprise adjusting said program depending on said comparison of said second blood pressure reading to said first blood pressure reading, wherein if said second blood pressure reading is not appreciably improved from said first blood pressure reading, directing said subject to continue ingesting a dose of said melatonin receptor agonist on a regular basis at night time for a second period.

In certain embodiments, the melatonin receptor agonist in the aforementioned methods is a compound of formula I, II, III, or IV.

In certain embodiments, the melatonin receptor agonist in the aforementioned methods is one or more of the following: N-[2-(5-Methoxy-1H-indol-3-yl)ethyl]acetamide (TAK-375), N-[2-(3-ethyl-7-methoxynaphthyl)ethyl]-acetamide (S21634), N-[2-(7-methoxynaphth-1-yl)-ethyl]-acetamide (S20098), N-[2-naphth-1-yl-ethyl]-cyclobutyl carboxamide (S20928), 2-iodomelatonin, N-acetyl-5-HT, LY 156735, BMS-214778, melatonin, agomelatine, CGP 52608, low-dose melatonin A, GR196429, S20242, S23478, S24268, S25150, melatonin receptor research compound A, GW290569, controlled release melatonin, luzindole, GR135531, melatonin agonist A, melatonin analogue B, melatonin agonist C, melatonin agonist D, melatonin agonist E, melatonin agonist F, melatonin agonist G, melatonin agonist H, melatonin agonist I, melatonin analog J , melatonin analog K, melatonin analog L, AH-001, GG-012, enol-3-IPA, ML-23, SL-18.1616, IP-100-9, melatonin low-dose B, sleep inducing peptide A, oros-melatonin, AH-017, AH-002, IP-101, 5-hydroxy-N-acetyl-tryptamine (NAT), 5-methoxy-N-bu-tanoyltryptamine (bMT), prazosin, phenylmelatonin, seradrene, P-methyl-6-chloromelatonin, 5-hydroxyethoxy-N-acetyltryptamine (5-HEAT), 8-methoxy-2-propionamidotetralin, PD-6735, seroctin, N-[2-(5-methoxy-2-phenylfuro[2,3-b]pyridin-3-yl)ethyl]acetamide, N-[2-(5-methoxy-2-phenylfuro[2,3-c]pyridin-3-yl)ethyl]acetamide, N-[(±)-2-(7-methoxy-1,2,3,4-tetrahydro-1-naphthyl)ethyl]cyclopropyl-carboxamide, N-[2-(7-methoxy-1-naphthyl)ethyl]acetamide, N-acetyl-4-aminomethyl-6-methoxy-9-methyl-1,2,3,4-tetrahydrocarbazole (AMMTC), 3-(2-aminopropyl)indole, 6-chloromelatonin, 2,3-dihydromelatonin, 6-chloro-2,3-dihydromelatonin, N-acetyl-N′-formyl-5-methoxykynurenamine, 6-methoxybenzoxazolinone, or a pharmaceutically acceptable salt, solvate, clathrate, polymorph, or co-crystal thereof.

In certain embodiments, the melatonin receptor agonist in the aforementioned methods is one or more of the following: N-[2-(5-Methoxy-1H-indol-3-yl)ethyl]acetamide (TAK-375), N-[2-(3-ethyl-7-methoxynaphthyl)ethyl]-acetamide (S21634), N-[2-(7-methoxynaphth-1-yl)-ethyl]-acetamide (S20098), N-[2-naphth-1-yl-ethyl]-cyclobutyl carboxamide (S20928), 2-iodomelatonin, N-acetyl-5-HT, LY 156735, BMS-214778, melatonin, or a pharmaceutically acceptable salt, solvate, clathrate, polymorph, or co-crystal thereof.

### BRIEF DESCRIPTION OF THE DRAWINGS

FIG. 1 shows a schematic of the study design. After randomization, patients started with the two single applications followed by the two 3 week applications. Arrows indicate time of ambulatory blood pressure measurements.

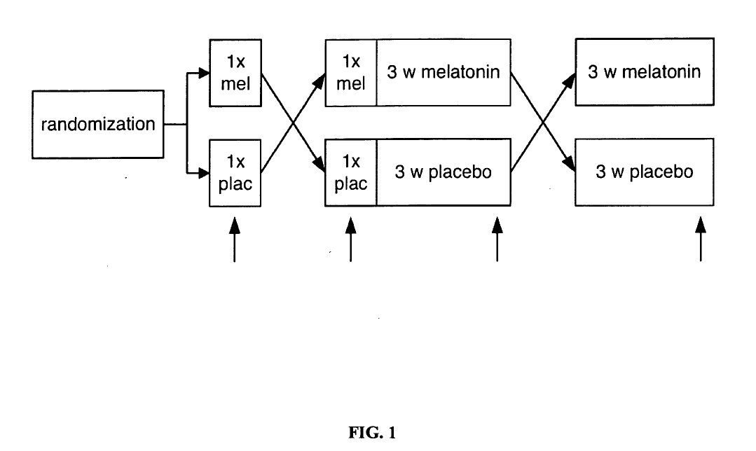

FIGS. 2A-C show graphs of the effect of repeated melatonin on individual sleep and wake blood pressure and heart rate. Zero values indicate the levels assessed after placebo. The effect of repeated melatonin of patient 14 was −53 and −27 mm Hg, for waking systolic and diastolic blood pressure, respectively. Dark bars indicate sleep and light bars indicate wake.

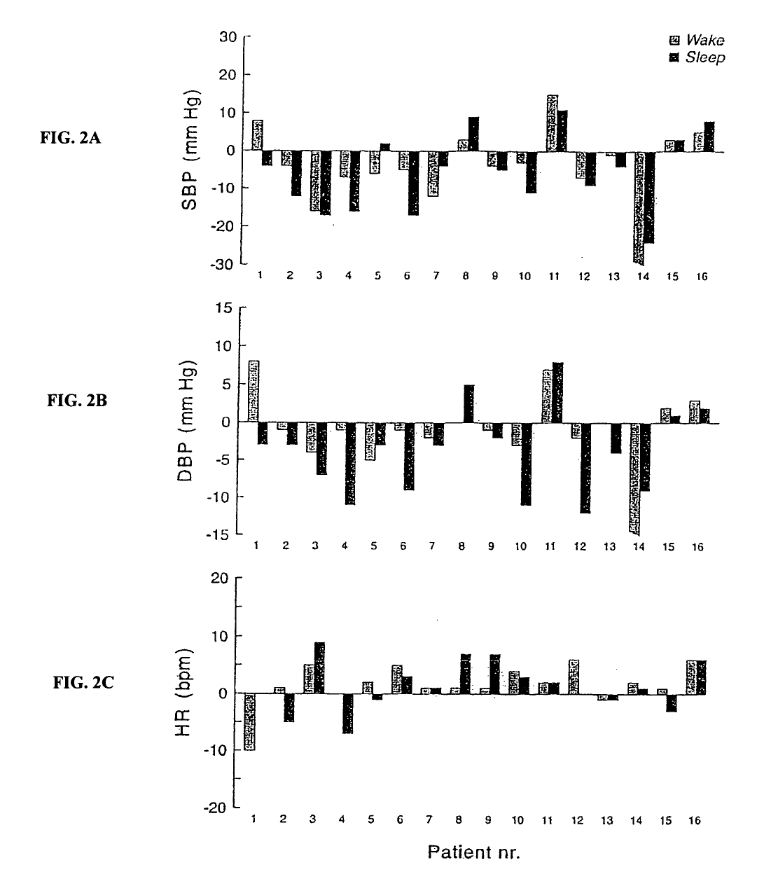

FIG. 3 shows a graph of the hourly means of ambulatory blood pressure after repeated melatonin and repeated placebo. The average systolic and diastolic ambulatory blood pressure of all patients is depicted relative to get up time (n=16). The light gray background indicates the average period in bed (7.1 hours±44 minutes). The vertical dotted line indicates the get up time. Open circles indicate placebo and closed circles indicate melatonin.

FIG. 4 shows a graph representing the twenty four hour fit of systolic and diastolic blood pressure rhythm. The average peaked and skewed cosine fit for systolic (n=14) and diastolic (n=1 5) ambulatory blood pressure of all patients with a significant fit is depicted relative to clock time. Circular statistics were used for the analysis of differences in bathyphase (time of minimum) between the placebo and melatonin condition in blood pressure (Mardia-Watson-Wheeler Chi²test; E. Batschelet, Circular Statistics in Biology, Academic Press, 1981, p. 371).

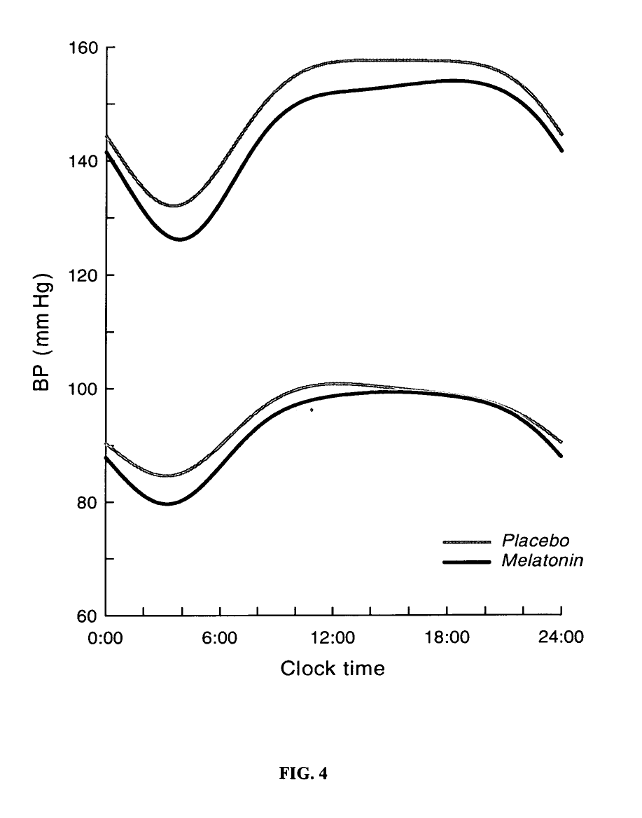

### DETAILED DESCRIPTION

The present disclosure provides methods and compositions for treating hypertension. Patients with essential hypertension have disturbed autonomic cardiovascular regulation and circadian pacemaker function. We conducted a randomized, double-blind, placebo-controlled, crossover trial in sixteen men with untreated essential hypertension to investigate the influence of acute (single) and repeated (3 weeks daily) oral melatonin (2.5 mg) intake 1 hour before sleep on 24-hour ambulatory blood pressure and actigraphic estimates of sleep quality. We have now suprisingly found that repeated melatonin intake reduces systolic and diastolic blood pressure during sleep by about 6 and 4 mm Hg, respectively. The treatment did not affect heart rate. The day-night amplitude of the rhythms in both systolic and diastolic blood pressure were increased by 15% and 25%, respectively. A single dose of melatonin had no effect on blood pressure. Repeated (but not acute) melatonin also improved sleep efficiency. Improvements in blood pressure and sleep were statistically unrelated.

Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The terms “comprise” and “comprising” are used in the inclusive, open sense, meaning that additional elements may be included.

The term “including” is used to mean “including but not limited to”. “Including” and “including but not limited to” are used interchangeably.

The term “melatonin receptor agonist” refers to a compound that binds to at least one receptor for melatonin, including for example, the melatonin 1a receptor and/or the melatonin 1b receptor. In one embodiment, melatonin receptor agonists exhibit a blood pressure lowering effect when administered to an animal that is similar to, or greater than, the blood pressure lowering effect of melatonin itself. Non-limiting examples of melatonin receptor agonists include compounds of Formula I, II, III, or IV. Non-limiting examples of melatonin receptor agonists further include: N-[2-(5-Methoxy-1H-indol-3-yl)ethyl]acetamide (TAK-375), N-[2-(3-ethyl-7-methoxynaphthyl)ethyl]-acetamide (e.g., S21634), N-[2-(7-methoxynaphth-1-yl)-ethyl]-acetamide (S20098), N-[2-naphth-1-yl-ethyl]-cyclobutyl carboxamide (S20928), 2-iodomelatonin, N-acetyl-5-HT, LY 156735, or BMS-214778. In certain embodiments, the term melatonin receptor agonist may include or exclude melatonin itself. Melatonin receptor agonists, or compositions comprising a melatonin receptor agonist, are also referred to herein as “antihypertensive agents,” “antihypertensive compositions,” and “melatonin analogs.”

The term “binding” refers to an association, which may be a stable association, between two molecules, e.g., between a melatonin receptor agonist and a melatonin receptor, due to, for example, electrostatic, hydrophobic, ionic and/or hydrogen-bond interactions under physiological conditions.

The term “essential hypertension” refers to systemic hypertension of an unknown cause. The term “secondary hypertension” refers to systemic hypertension of a known and reversible cause, such as, for example, renal artery diseases or endocrine disorders. The terms essential hypertension and secondary hypertension are meant to encompass hypertension of all grades, including borderline, mild moderate and severe.

The phrase “blood pressure lowering amount” refers to that amount of a melatonin receptor agonist that produces a therapeutically effective decrease in the blood pressure of a subject. In certain embodiments, a therapeutically effective decrease in blood pressure refers to a reduction of at least about 2 mm Hg of systolic and/or diastolic blood pressure relative to either the subject's normal base line blood pressure or the subject's blood pressure under placebo therapy.

The term “cis” is art-recognized and refers to the arrangement of two atoms or groups around a double bond such that the atoms or groups are on the same side of the double bond. Cis configurations are often labeled as (Z) configurations.

The term “trans” is art-recognized and refers to the arrangement of two atoms or groups around a double bond such that the atoms or groups are on the opposite sides of a double bond. Trans configurations are often labeled as (E) configurations.

The term “covalent bond” is art-recognized and refers to a bond between two atoms where electrons are attracted electrostatically to both nuclei of the two atoms, and the net effect of increased electron density between the nuclei counterbalances the internuclear repulsion. The term covalent bond includes coordinate bonds when the bond is with a metal ion.

The term “therapeutic agent” is art-recognized and refers to any chemical moiety that is a biologically, physiologically, or pharmacologically active substance that acts locally or systemically in a subject. Examples of therapeutic agents, also referred to as “drugs”, are described in well-known literature references such as the Merck Index, the Physicians Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment.

The term “therapeutic effect” is art-recognized and refers to a local or systemic effect in animals, particularly mammals, and more particularly humans caused by a pharmacologically active substance. The term thus means any substance intended for use in the diagnosis, cure, mitigation, treatment or prevention of disease or in the enhancement of desirable physical or mental development and/or conditions in an animal or human. The phrase “therapeutically-effective amount” means that amount of such a substance that produces some desired local or systemic effect at a reasonable benefit/risk ratio applicable to any treatment. The therapeutically effective amount of such substance will vary depending upon the subject and disease condition being treated, the weight and age of the subject, the severity of the disease condition, the manner of administration and the like, which can readily be determined by one of ordinary skill in the art. For example, certain compositions of the present invention may be administered in a sufficient amount to produce a at a reasonable benefit/risk ratio applicable to such treatment.

The term “synthetic” is art-recognized and refers to production by in vitro chemical or enzymatic synthesis.

The term “meso compound” is art-recognized and refers to a chemical compound which has at least two chiral centers but is achiral due to a plane or point of symmetry.

The term “chiral” is art-recognized and refers to molecules which have the property of non-superimposability of the mirror image partner, while the term “achiral” refers to molecules which are superimposable on their mirror image partner. A “prochiral molecule” is a molecule which has the potential to be converted to a chiral molecule in a particular process.

The term “stereoisomers” is art-recognized and refers to compounds which have identical chemical constitution, but differ with regard to the arrangement of the atoms or groups in space. In particular, “enantiomers” refer to two stereoisomers of a compound which are non-superimposable mirror images of one another. “Diastereomers”, on the other hand, refers to stereoisomers with two or more centers of dissymmetry and whose molecules are not mirror images of one another.

Furthermore, a “stereoselective process” is one which produces a particular stereoisomer of a reaction product in preference to other possible stereoisomers of that product. An “enantioselective process” is one which favors production of one of the two possible enantiomers of a reaction product.

The term “regioisomers” is art-recognized and refers to compounds which have the same molecular formula but differ in the connectivity of the atoms. Accordingly, a “regioselective process” is one which favors the production of a particular regioisomer over others, e.g., the reaction produces a statistically significant increase in the yield of a certain regioisomer.

The term “epimers” is art-recognized and refers to molecules with identical chemical constitution and containing more than one stereocenter, but which differ in configuration at only one of these stereocenters.

The term “ED₅₀” is art-recognized. In certain embodiments, ED₅₀ means the dose of a drug which produces 50% of its maximum response or effect, or alternatively, the dose which produces a pre-determined response in 50% of test subjects or preparations. The term “LD₅₀” is art-recognized. In certain embodiments, LD₅₀ means the dose of a drug which is lethal in 50% of test subjects. The term “therapeutic index” is an art-recognized term which refers to the therapeutic index of a drug, defined as LD₅₀/ED₅₀.

The term “prodrug” is art-recognized and is intended to encompass compounds which, under physiological conditions, are converted into the antihypertensive agents of the present invention. A common method for making a prodrug is to select moieties which are hydrolyzed under physiological conditions to provide the desired compound. In other embodiments, the prodrug is converted by an enzymatic activity of the host animal.

The term “structure-activity relationship” or “(SAR)” is art-recognized and refers to the way in which altering the molecular structure of a drug or other compound alters its interaction with a receptor, enzyme, nucleic acid or other target and the like.

The term “aliphatic” is art-recognized and refers to a linear, branched, cyclic alkane, alkene, or alkyne. In certain embodiments, aliphatic groups in the present invention are linear or branched and have from 1 to about 20 carbon atoms.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure. The term “alkyl” is also defined to include halosubstituted alkyls.

The term “aralkyl” is art-recognized and refers to an alkyl group substituted with an aryl group (e.g., an aromatic or heteroaromatic group).

The terms “alkenyl” and “alkynyl” are art-recognized and refer to unsaturated aliphatic groups analogous in length and possible substitution to the alkyls described above, but that contain at least one double or triple bond respectively.

Unless the number of carbons is otherwise specified, “lower alkyl” refers to an alkyl group, as defined above, but having from one to about ten carbons, alternatively from one to about six carbon atoms in its backbone structure. Likewise, “lower alkenyl” and “lower alkynyl” have similar chain lengths.

The term “heteroatom” is art-recognized and refers to an atom of any element other than carbon or hydrogen. Illustrative heteroatoms include boron, nitrogen, oxygen, phosphorus, sulfur and selenium.

The term “aryl” is art-recognized and refers to 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “heteroaryl.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

The terms “heterocyclyl” or “heterocyclic group” are art-recognized and refer to 3- to about 10-membered ring structures, alternatively 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxanthene, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” are art-recognized and refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle” is art-recognized and refers to an aromatic or non-aromatic ring in which each atom of the ring is carbon.

The term “nitro” is art-recognized and refers to —NO₂; the term “halogen” is art-recognized and refers to —F, —Cl, —Br or —I; the term “sulfhydryl” is art-recognized and refers to —SH; the term “hydroxyl” means —OH; and the term “sulfonyl” is art-recognized and refers to —SO₂⁻. “Halide” designates the corresponding anion of the halogens, and “pseudohalide” has the definition set forth on page 560 of “Advanced Inorganic Chemistry” by Cotton and Wilkinson.

The terms “amine” and “amino” are art-recognized and refer to both unsubstituted and substituted amines, e.g., a moiety that may be represented by the general formulas: 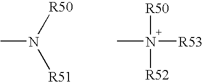
wherein R50, R51 and R52 each independently represent a hydrogen, an alkyl, an alkenyl, —(CH₂)ₘ—R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In certain embodiments, only one of R50 or R51 may be a carbonyl, e.g., R50, R51 and the nitrogen together do not form an imide. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group.

The term “acylamino” is art-recognized and refers to a moiety that may be represented by the general formula: 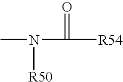
wherein R50 is as defined above, and R54 represents a hydrogen, an alkyl, an alkenyl or —(CH₂)ₘ—R61, where m and R61 are as defined above.

The term “amido” is art recognized as an amino-substituted carbonyl and includes a moiety that may be represented by the general formula: 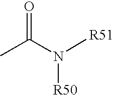
wherein R50 and R51 are as defined above. Certain embodiments of the amide in the present invention will not include imides which may be unstable.

The term “alkylthio” refers to an alkyl group, as defined above, having a sulfur radical attached thereto. In certain embodiments, the “alkylthio” moiety is represented by one of —S-alkyl, —S-alkenyl, —S-alkynyl, and —S-(CH₂)ₘ—R61, wherein m and R61 are defined above. Representative alkylthio groups include methylthio, ethyl thio, and the like.

The term “carbonyl” is art recognized and includes such moieties as may be represented by the general formulas: 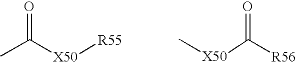
wherein X50 is a bond or represents an oxygen or a sulfur, and R55 and R56 represents a hydrogen, an alkyl, an alkenyl, —(CH₂)ₘ—R61 or a pharmaceutically acceptable salt, R56 represents a hydrogen, an alkyl, an alkenyl or —(CH₂)ₘ—R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiolcarbonyl” group. Where X50 is a sulfur and R55 or R56 is not hydrogen, the formula represents a “thiolester.” Where X50 is a sulfur and R55 is hydrogen, the formula represents a “thiolcarboxylic acid.” Where X50 is a sulfur and R56 is hydrogen, the formula represents a “thiolformate.” On the other hand, where X50 is a bond, and R55 is not hydrogen, the above formula represents a “ketone” group. Where X50 is a bond, and R55 is hydrogen, the above formula represents an “aldehyde” group.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R61, where m and R61 are described above.

The term “sulfonate” is art recognized and refers to a moiety that may be represented by the general formula: 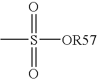
in which R57 is an electron pair, hydrogen, alkyl, cycloalkyl, or aryl.

The term “sulfate” is art recognized and includes a moiety that may be represented by the general formula: 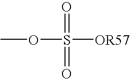
in which R57 is as defined above.

The term “sulfonamido” is art recognized and includes a moiety that may be represented by the general formula: 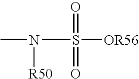
in which R50 and R56 are as defined above.

The term “sulfamoyl” is art-recognized and refers to a moiety that may be represented by the general formula: 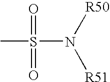
in which R50 and R51 are as defined above.

The term “sulfonyl” is art-recognized and refers to a moiety that may be represented by the general formula: 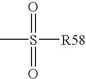
in which R58 is one of the following: hydrogen, alkyl, alkenyl, alkynyl, cycloalkyl, heterocyclyl, aryl or heteroaryl.

The term “sulfoxido” is art-recognized and refers to a moiety that may be represented by the general formula: 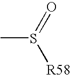
in which R58 is defined above.

The term “phosphoryl” is art-recognized and may in general be represented by the formula: 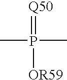
wherein Q50 represents S or O, and R59 represents hydrogen, a lower alkyl or an aryl. When used to substitute, e.g., an alkyl, the phosphoryl group of the phosphorylalkyl may be represented by the general formulas: 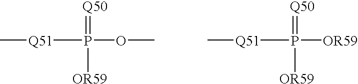
wherein Q50 and R59, each independently, are defined above, and Q51 represents O, S or N. When Q50 is S, the phosphoryl moiety is a “phosphorothioate”.

The term “phosphoramidite” is art-recognized and may be represented in the general formulas: 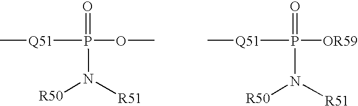
wherein Q51, R50, R51 and R59 are as defined above.

The term “phosphonamidite” is art-recognized and may be represented in the general formulas: 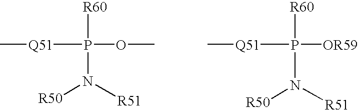
wherein Q51, R50, R51 and R59 are as defined above, and R60 represents a lower alkyl or an aryl.

Analogous substitutions may be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

The definition of each expression, e.g. alkyl, m, n, and the like, when it occurs more than once in any structure, is intended to be independent of its definition elsewhere in the same structure.

The term “selenoalkyl” is art-recognized and refers to an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)ₘ—R61, m and R61 being defined above.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

Certain compounds contained in compositions of the present invention may exist in particular geometric or stereoisomeric forms. In addition, polymers of the present invention may also be optically active. The present invention contemplates all such compounds, including cis- and trans-isomers, R—and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

For purposes of this invention, the chemical elements are identified in accordance with the Periodic Table of the Elements, CAS version, Handbook of Chemistry and Physics, 67th Ed., 1986-87, inside cover. Also for purposes of this invention, the term “hydrocarbon” is contemplated to include all permissible compounds having at least one hydrogen and one carbon atom. In a broad aspect, the permissible hydrocarbons include acyclic and cyclic, branched and unbranched, carbocyclic and heterocyclic, aromatic and nonaromatic organic compounds that may be substituted or unsubstituted.

The term “protecting group” is art-recognized and refers to temporary substituents that protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed by Greene and Wuts in Protective Groups in Organic Synthesis (2^{nd} ed., Wiley: New York, 1991).

The term “hydroxyl-protecting group” is art-recognized and refers to those groups intended to protect a hydrozyl group against undesirable reactions during synthetic procedures and includes, for example, benzyl or other suitable esters or ethers groups known in the art.

The term “carboxyl-protecting group” is art-recognized and refers to those groups intended to protect a carboxylic acid group, such as the C-terminus of an amino acid or peptide or an acidic or hydroxyl azepine ring substituent, against undesirable reactions during synthetic procedures and includes. Examples for protecting groups for carboxyl groups involve, for example, benzyl ester, cyclohexyl ester, 4-nitrobenzyl ester, t-butyl ester, 4-pyridylmethyl ester, and the like.

The term “amino-blocking group” is art-recognized and refers to a group which will prevent an amino group from participating in a reaction carried out on some other functional group, but which can be removed from the amine when desired. Such groups are discussed by in Ch. 7 of Greene and Wuts, cited above, and by Barton, Protective Groups in Organic Chemistry ch. 2 (McOmie, ed., Plenum Press, New York, 1973). Examples of suitable groups include acyl protecting groups such as, to illustrate, formyl, dansyl, acetyl, benzoyl, trifluoroacetyl, succinyl, methoxysuccinyl, benzyl and substituted benzyl such as 3,4-dimethoxybenzyl, o-nitrobenzyl, and triphenylmethyl; those of the formula —COOR where R includes such groups as methyl, ethyl, propyl, isopropyl, 2,2,2-trichloroethyl, 1-methyl-1-phenylethyl, isobutyl, t-butyl, t-amyl, vinyl, allyl, phenyl, benzyl, p-nitrobenzyl, o-nitrobenzyl, and 2,4-dichlorobenzyl; acyl groups and substituted acyl such as formyl, acetyl, chloroacetyl, dichloroacetyl, trichloroacetyl, trifluoroacetyl, benzoyl, and p-methoxybenzoyl; and other groups such as methanesulfonyl, p-toluenesulfonyl, p-bromobenzenesulfonyl, p-nitrophenylethyl, and p-toluenesulfonyl-aminocarbonyl. Preferred amino-blocking groups are benzyl (—CH₂C₆H₅), acyl [C(O)R1] or SiR1₃ where R1 is C₁-C₄ alkyl, halomethyl, or 2-halo-substituted-(C₂-C₄ alkoxy), aromatic urethane protecting groups as, for example, carbonylbenzyloxy (Cbz); and aliphatic urethane protecting groups such as t-butyloxycarbonyl (Boc) or 9-fluorenylmethoxycarbonyl (FMOC).

The definition of each expression, e.g. lower alkyl, m, n, p and the like, when it occurs more than once in any structure, is intended to be independent of its definition elsewhere in the same structure.

The term “electron-withdrawing group” is art-recognized, and refers to the tendency of a substituent to attract valence electrons from neighboring atoms, i.e., the substituent is electronegative with respect to neighboring atoms. A quantification of the level of electron-withdrawing capability is given by the Hammett sigma (a) constant. This well known constant is described in many references, for instance, March, Advanced Organic Chemistry 251-59 (McGraw Hill Book Company: New York, 1977). The Hammett constant values are generally negative for electron donating groups (σ(P)=−0.66 for NH₂) and positive for electron withdrawing groups (σ(P)=0.78 for a nitro group), σ(P) indicating para substitution. Exemplary electron-withdrawing groups include nitro, acyl, formyl, sulfonyl, trifluoromethyl, cyano, chloride, and the like. Exemplary electron-donating groups include amino, methoxy, and the like.

The term “small molecule” is art-recognized and refers to a composition which has a molecular weight of less than about 2000 amu, or less than about 1000 amu, and even less than about 500 amu. Small molecules may be, for example, nucleic acids, peptides, polypeptides, peptide nucleic acids, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays of the invention. The term “small organic molecule” refers to a small molecule that is often identified as being an organic or medicinal compound, and does not include molecules that are exclusively nucleic acids, peptides or polypeptides.

A “target” shall mean a site to which targeted constructs bind. A target may be either in vivo or in vitro. In certain embodiments, a target may be a molecular structure to which a targeting moiety binds, such as a hapten, epitope, receptor, dsDNA fragment, carbohydrate or enzyme.

The term “modulation” is art-recognized and refers to up regulation (i.e., activation or stimulation), down regulation (i.e., inhibition or suppression) of a response, or the two in combination or apart.

The term “treating” is art-recognized and refers to curing as well as ameliorating at least one symptom of any condition or disease. In an exemplary embodiment, treating relates to any treatment of a hypertensive disease, including, for example (1) preventing hypertension from occurring in a subject who may be predisposed to the disease but who has not yet been diagnosed as having it; (2) inhibiting the disease, i.e., arresting its development; or (3) ameliorating or relieving the symptoms of the disease, i.e., causing regression of the hypertensive state.

The term “prophylactic” or “therapeutic” treatment is art-recognized and refers to administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

A “patient,” “subject” or “host” to be treated by the subject method may mean either a human or non-human animal.

The term “mammal” is known in the art, and exemplary mammals include humans, primates, bovines, porcines, canines, felines, and rodents (e.g., mice and rats).

The term “bioavailable” is art-recognized and refers to a form of the subject invention that allows for it, or a portion of the amount administered, to be absorbed by, incorporated to, or otherwise physiologically available to a subject or patient to whom it is administered.

The term “pharmaceutically-acceptable salts” is art-recognized and refers to the relatively non-toxic, inorganic and organic acid addition salts of compounds, including, for example, those contained in compositions of the present invention.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof from one organ, or portion of the body, to another organ, or portion of the body. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The terms “systemic administration,” “administered systemically,” “peripheral administration” and “administered peripherally” are art-recognized and refer to the administration of a subject composition, therapeutic or other material other than directly into the central nervous system, such that it enters the patient's system and, thus, is subject to metabolism and other like processes, for example, subcutaneous administration.

The terms “parenteral administration” and “administered parenterally” are art-recognized and refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articulare, subcapsular, subarachnoid, intraspinal, and intrasternal injection and infusion.

Contemplated equivalents of the compositions described herein include compositions which otherwise correspond thereto, and which have the same general properties thereof, wherein one or more simple variations of substituents or components are made which do not adversely affect the characteristics of the compositions of interest. In general, the components of the compositions of the present invention may be prepared by the methods illustrated in the general reaction schema as, for example, described below, or by modifications thereof, using readily available starting materials, reagents and conventional synthesis procedures. In these reactions, it is also possible to make use of variants which are in themselves known, but are not mentioned here.

Methods

In one embodiment, the present invention relates to a method of treating hypertension in a patient comprising administering to the patient a compound of formula I: 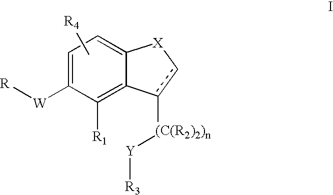
wherein, independently for each occurrence:
R is C₁₋₆ alkyl, C₂₋₈ alkenyl, C₂₋₈ alkynyl, cycloalkyl, aryl, aralkyl, heteroaryl, heteroaralkyl, —CO₂(C₁₋₆ alkyl), —CO₂(aryl), —C(O)NH(C₁₋₆ alkyl), —C(O)NH(aryl), —C(O)H, —C(O)(aryl), or —C(O)(C₁₋₆ alkyl);
R₁ is H, C₁₋₆ alkyl, C₂₋₈ alkenyl, C₂₋₈ alkynyl, aryl, aralkyl, heteroaryl, heteroaralkyl; or R and R₁ taken together form a fused aromatic, cycloalkyl, or cycloalkenyl ring;

R₂ is H, C₁₋₆ alkyl, cycloalkyl, aryl, aralkyl, or —CO₂R₁;
R₃ is H, C₁₋₆ alkyl, cycloalkyl, aryl, aralkyl, or —C(O)R₁;
R₄ is H, C₁₋₆ alkyl, or halide;
R₅ is H, C₁₋₆ alkyl, C₁₋₆ alkoxy; aryl, aralkyl, cycloalkyl, —N(R₁)₂, or heteroaryl;
W is O, N(R₂), or S;
X is CH₂, O, N(R₂) or S;
Y is O, N(R₆), or S;
R₆ is H, C₁₋₆ alkyl, —CO₂(C₁₋₆ alkyl); or R₆ and R₃ taken together form a ring;
the 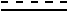
   line indicates either a single or double bond between the two carbon atoms; and
n is an integer from 1 to 6 inclusive;
or a pharmaceutically acceptable salt, solvate, clathrate, polymorph, or co-crystal thereof.

In certain embodiments, the present invention relates to a method of treating hypertension in a patient comprising administering to the patient a compound of formula II: 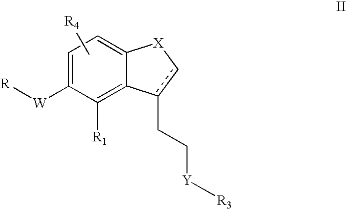
wherein, independently for each occurrence:
R is C₁₋₆ alkyl, C₂₋₈ alkenyl, C₂₋₈ alkynyl, cycloalkyl, aryl, aralkyl, heteroaryl, heteroaralkyl, —CO₂(C₁₋₆ alkyl), —CO₂(aryl), —C(O)NH(C₁₋₆ alkyl), —C(O)NH(aryl), —C(O)H, —C(O)(aryl), or —C(O)(C₁₋₆ alkyl);
R₁ is H, C₁₋₆ alkyl, C₂₋₈ alkenyl, C₂₋₈ alkynyl, aryl, aralkyl, heteroaryl, heteroaralkyl; or R and R₁ taken together form a fused aromatic, cycloalkyl, or cycloalkenyl ring;
R₃ is H, C₁₋₆ alkyl, cycloalkyl, aryl, aralkyl, or —C(O)R₁;
R₄ is H, C₁₋₆ alkyl, or halide;
W is O or S;
X is CH₂, O, N(R₁) or S;
Y is O, N(R₆), or S;
R₆ is H, C₁₋₆ alkyl, —CO₂(C₁₋₆ alkyl); or R₆ and R₃ taken together form a ring; and
the 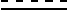
   line indicates either a single or double bond between the two carbon atoms;
or a pharmaceutically acceptable salt, solvate, clathrate, polymorph, or co-crystal thereof.

In certain embodiments, the present invention relates to a method of treating hypertension in a patient comprising administering to the patient a compound of formula III: 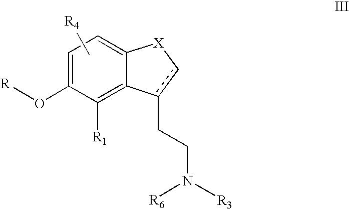
wherein, independently for each occurrence:
R is C₁₋₆ alkyl, C₂₋₈ alkenyl, C₂₋₈ alkynyl, cycloalkyl, aryl, aralkyl, heteroaryl, heteroaralkyl, —CO₂(C₁₋₆ alkyl), —CO₂(aryl), —C(O)NH(C₁₋₆ alkyl), —C(O)NH(aryl), —C(O)H, —C(O)(aryl), or —C(O)(C₁₋₆ alkyl);
R₁ is H, C₁₋₆ alkyl, C₂₋₈ alkenyl, C₂₋₈ alkynyl, aryl, aralkyl, heteroaryl, heteroaralkyl; or R and R₁ taken together form a fused aromatic, cycloalkyl, or cycloalkenyl ring;
R₃ is H, C₁₋₆ alkyl, cycloalkyl, aryl, aralkyl, or —C(O)R₁;
R₄ is H, C₁₋₆ alkyl, or halide;
X is CH₂ or N(R₁);
R₆ is H, C₁₋₆ alkyl, —CO₂(C₁₋₆ alkyl); or R₆ and R₃ taken together form a ring; and
the 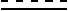
   line indicates either a single or double bond between the two carbon atoms;
or a pharmaceutically acceptable salt, solvate, clathrate, polymorph, or co-crystal thereof.

In certain embodiments, the present invention relates to a method of treating hypertension in a patient comprising administering to the patient a compound of formula IV: 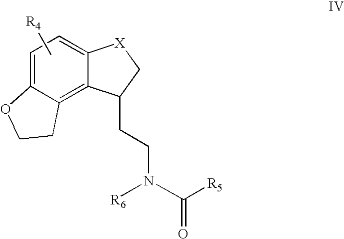
wherein, independently for each occurrence:
R₄ is H, C₁₋₆ alkyl, or halide;
R₅ is H, C₁₋₆ alkyl, C₁₋₆ alkoxy; aryl, aralkyl, cycloalkyl, or heteroaryl;
X is CH₂ or N(R₅); and
R₆ is H, C₁₋₆ alkyl, —CO₂(C₁₋₆ alkyl);
or a pharmaceutically acceptable salt, solvate, clathrate, polymorph, or co-crystal thereof.

In certain embodiments, the present invention relates to a method of treating hypertension in a patient comprising administering to the patient a melatonin receptor agonist wherein the melatonin receptor agonist is one or more of the following: N-[2-(5-Methoxy-1H-indol-3-yl)ethyl]acetamide (TAK-375), N-[2-(3-ethyl-7-methoxynaphthyl)ethyl]-acetamide (S21634), N-[2-(7-methoxynaphth-1-yl)-ethyl]-acetamide (S20098), N-[2-naphth-1-yl-ethyl]-cyclobutyl carboxamide (S20928), 2-iodomelatonin, N-acetyl-5-HT, LY 156735, BMS-214778, melatonin, agomelatine, CGP 52608, low-dose melatonin A, GR196429, S20242, S23478, S24268, S25150, melatonin receptor research compound A, GW290569, controlled release melatonin, luzindole, GR135531, melatonin agonist A, melatonin analogue B, melatonin agonist C, melatonin agonist D, melatonin agonist E, melatonin agonist F, melatonin agonist G, melatonin agonist H, melatonin agonist I, melatonin analog J, melatonin analog K, melatonin analog L, AH-001, GG-012, enol-3-IPA, ML-23, SL-18.1616, IP-1 00-9, melatonin low-dose B, sleep inducing peptide A, oros-melatonin, AH-017, AH-002, IP-101, 5-hydroxy-N-acetyl-tryptamine (NAT), 5-methoxy-N-bu-tanoyltryptamine (bMT), prazosin, phenylmelatonin, seradrene, β-methyl-6-chloromelatonin, 5-hydroxyethoxy-N-acetyltryptamine (5-HEAT), 8-methoxy-2-propionamidotetralin, PD-6735, seroctin, N-[2-(5-methoxy-2-phenylfuro[2,3-b]pyridin-3-yl)ethyl]acetamide, N-[2-(5-methoxy-2-phenylfuro[2,3-c]pyridin-3-yl)ethyl]acetamide, N-[(±)-2-(7-methoxy-1,2,3,4-tetrahydro-1-naphthyl)ethyl]cyclopropyl-carboxamide, N-[2-(7-methoxy-1-naphthyl)ethyl]acetamide, N-acetyl-4-aminomethyl-6-methoxy-9-methyl-1,2,3,4-tetrahydrocarbazole (AMMTC), 3-(2-aminopropyl)indole, 6-chloromelatonin, 2,3-dihydromelatonin, 6-chloro-2,3-dihydromelatonin, N-acetyl-N′-formyl-5-methoxykynurenamine, 6-methoxybenzoxazolinone, or a pharmaceutically acceptable salt, solvate, clathrate, polymorph, or co-crystal thereof. See e.g., Tuma J, et al., Chronobiol Int. 18(5):781-99 (2001); Conway S, et al., Biochem Biophys Res Commun. 282(5):1229-36 (2001); Weibel Van Reeth O, et al., Am J Physiol Regul Integr Comp Physiol. 280(5):R1582-91 (2001); Weibel L, et al., Brain Res. 880(1-2):207-11 (2000); Ting N, et al., Naunyn Schmiedebergs Arch Pharmacol. 361(3):327-33 (2000); Loo H, et al., Encephale. 29(2):165-71 (2003); Millan M J, et al., Pharmacol Exp Ther. 306(3):954-64 (2003); Descamps-Francois C, et al., J Med Chem. 46(7):1127-9 (2003); Loo H, et al., Int Clin Psychopharmacol. 17(5):239-47 (2002); Nickelsen T, et al., Chronobiol Int. 19(5):915-36 (2002); Mohamed Naguib, et al., Anesth Analg 97:763-768 (2003); Vachharajani N N, et al., J Pharm Sci. 92(4):760-72 (2003); Mattson R J, et al., Bioorg Med Chem Lett. 13(6):1199-202 (2003); Patel R N, Curr Opin Biotechnol. 12(6):587-604 (2001); Uchikawa O, et al., J Med Chem. 45(19):4222-39 (2002); Barchas et al. Nature 1967, 214, 919; Kato, K. et al. Int. J. Neuropsychopharmacol. 2000, 3(Suppl. 1): Abst P.03.130; see also abstracts P.03.125 and P.03.127); Loo, H.; Hale, A., D'haenen, H. Int. Clin. Psychopharmacol. 2002, 17, 239-47; Nickelsen T, Samel A, Vejvoda M, Wenzel J, Smith B, Gerzer R. Chronobiol Int. 2002, 19, 915-36; Missbach, M.; Jagher, B.; Sigg, I.; Nayeri, S.; Carlberg, C.; Wiesenberg, I. J. Biol. Chem. 1996, 271, 13515-22; Wisenberg, I.,; Missbach, M.; Kahlen, J.-P.; Schrader, M.; Carlberg, C. Nuc. Acids Res. 1995, 23, 327-333; Beresford, I. J.; Browning, C; Starkey, S. J.; Brown, J; Foord, S. M.; Coughlan, J; North, P. C.; Dubocovich, M. L.; Hagan, R. M.; J. Pharmacol. Exp. Ther. 1998, 285, 1239-1245 and Cutler, D. J.; Beresford, I. J. M.; Mason, R. Pharmacologist 1997, 39, 118; Depres-Brummer P, Metzger G, Levi F. Eur. J. Pharmacol. 1998, 347, 57-66 and Koster-van Hoffen, G. C.; Mirmiran, M.; Bos, N. P.; Witting, W.; Delagrange, P.; Guardiola-Lemaitre, B. Neurobiol Aging. 1993, 14, 565-9; Neuropharmacology July 2000; Naunyn Schmiedebergs Arch. 6/03; Vachharajani, N. N.; Yeleswaram, K.; Boulton, D. W. J. Pharm. Sci. 2003, 92, 760-72; Drugs R&D 2003, Adis R&D December 2002; Decision Resources October 1996; Dubocovich, M. L. J. Pharmacol. Exp. Ther. 1988, 246, 902; Beresford, I. J.; Harvey, F. J.; Hall, D. A.; Giles, H. Biochem Pharmacol. 1998, 56, 1167-74; IMSWorld R&D Focus August 2002; Pharmaprojects August 1998; Chem. Pharm. Bull. (Tokyo) January 2002; J. Pineal Research November 2000; Chem. Pharm. Bull. (Tokyo) Febrary 2002; Reprod. Nutr. Dev. May 1999; J. Med. Chem. October 1993; Famaco March 2000; J. Med. Chem. March 2000; Bioorg. Med. Chem. Lett. March 2003; MedAd News September 2001; Drijfhout, W. J. et al. Eur. J. Pharmacol. 1999, 382, 157-66; Drijfhout, W. J. et al. Eur. J. Pharmacol. 1999, 382, 157-66; Buzzell, G. R.; Menendez-Pelaez, A.; Troiani, M. E.; McNeill, M. E.; Reiter, R. J. J. Pineal. Res. 1990, 8, 229-35 and Nordio, M.; Vaughan, M. K.; Zisapel, N.; Migliaccio, S.; van Jaarsveld, A.; Reiter, R. J. Proceedings of the Society for Experimental Biology and Medicine 1989, 191, 321-325; Shah, J.; Langmuir, V.; Gupta, S. K. J Clin. Pharmacol. 1999, 39, 606-612. The above references are incorporated herein in their entirety.

In certain embodiments, the melatonin receptor agonist may also be the melatonin analogs as described in WO9517405; EP0447285; EP0527687; EP0530087; EP0591057; U.S. Pat. Nos. 6,638, 966; 6,552,064; 6,310,085; 5,985,293; 5,939,084; 4,997,845; Ucar et al., J. Med. Chem., 1998, 41, 1138-1145; and Garratt et al., J. Med. Chem., 2000, 43, 1050-1061, the contents of which are incorporated herein in their entirety.

In certain embodiments, the present invention relates to a method of treating hypertension in a patient comprising administering to the patient a melatonin receptor agonist wherein the compound is one or more of the following: N-[2-(5-Methoxy-1H-indol-3-yl)ethyl]acetamide (TAK-375), N-[2-(3-ethyl-7-methoxynaphthyl)ethyl]-acetamide (S21634), N-[2-(7-methoxynaphth-1-yl)-ethyl]-acetamide (S20098), N-[2-naphth-1-yl-ethyl]-cyclobutyl carboxamide (S20928), 2-iodomelatonin, N-acetyl-5-HT, LY 156735, BMS-214778, melatonin, or a pharmaceutically acceptable salt, solvate, clathrate, polymorph, or co-crystal thereof.

Also included in the anti-hypertensive methods of the present invention are pharmaceutically acceptable addition salts and complexes of the compounds of formula I, II, III, or IV. In cases wherein the compounds may have one or more chiral centers, unless specified, the present invention comprises each unique racemic compound, as well as each unique nonracemic compound.

In cases in which the compounds have unsaturated carbon-carbon double bonds, both the cis (Z) and trans (E) isomers are within the scope of this invention. In cases wherein inhibitors may exist in tautomeric forms, such as keto-enol tautomers, such as and 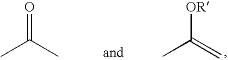
each tautomeric form is contemplated as being included within this invention, whether existing in equilibrium or locked in one form by appropriate substitution with R′. The meaning of any substituent at any one occurrence is independent of its meaning, or any other substituent's meaning, at any other occurrence.

Also included in the anti-hypertensive methods of the present invention are prodrugs of the compounds of formula I, II, III, or IV.

The compounds of formula I, II, III, or IV may be prepared by any conventional method useful for the preparation of analogous compounds. Starting materials for the processes are known or can be prepared by known processes from commercially available materials. A compound used in the methods of the present invention can be converted to another compound used in the methods of the present invention using conventional methods. The products of the reactions are isolated by conventional means such as extraction, crystallization, distillation, chromatography, and the like.

In certain embodiments, the melatonin receptor agonists described herein may be purchased from commercially available sources, such as, for example, Takeda (for example, TAK-375), Servier, Bristol-Meyers Squibb (for example, BMS-214778), and Eli Lilly and Company (for example LY 156735).

In certain embodiments, the antihypertensive agents described herein may be administered to a patient at night time on a daily basis for at leat two days. Adminstration at night time means that the antihypertensive agent is administered at, or near the bed time of the subject, such as for example, approximately 5 hours, 3 hours, 2 hours, 1 hour, 30 minutes, 20 minutes, 15 minutes, 10 minutes, 5 minutes, 2 minutes, or 1 minute prior to, or after, bed time, or during the night period (e.g., via a transdermal patch). In certain embodiments, administration of the antihypertensive agent may be at night time on a daily basis for at least about 2 days, 1, 2, 3, 4, 5, or 10 weeks, 3, 4, 5, 6, 8, 10, or 12 months, or 1, 2, 3, 4, 5, or more years.

In certain embodiments, it may be desirable to monitor the blood pressure of a subject receiving an antihypertensive agent as described herein before, during and/or after treatment with the subject antihypertensive agents. In exemplary embodiments, it may be desirable to monitor the blood pressure of a subject on a regular basis (e.g., hourly, daily, weekly, monthly, etc.) to monitor the course of treatment and/or deisgn a treatment strategy. Blood pressure may be monitoring using any technique known in the art, such as, for example, a Finometer (TNO Biomedical Instruments, Amsterdam, The Netherlands) or a sphygmometer.

In certain embodiments, the antihypertensive agents and compositions useful in the methods of the invention may be supplied with printed instructions which direct the user to employ the compositions in the methods and for the purposes described herein. Accordingly, said instructions are considered part of the present invention. The instructions for use may be printed on a container housing the composition or on a separate sheet which is included with the composition. Among other things, the instructions, may for example, direct the user to employ the composition and may also state that the purpose of such method is to inhibit or otherwise prevent symptoms of or associated with hypertension. The instructions may be directed to normotensive individuals who may be predisposed to hypertension and/or to those already diagnosed as having essential hypertension.

Formulations

The antihypertensive compositions of the present invention may be administered by various means, depending on their intended use, as is well known in the art. For example, if compositions of the present invention are to be administered orally, they may be formulated as tablets, capsules, granules, powders or syrups. Alternatively, formulations of the present invention may be administered parenterally as injections (intravenous, intramuscular, transdermal patch, by aerosols or subcutaneous), drop infusion preparations or suppositories. For application by the ophthalmic mucous membrane route, compositions of the present invention may be formulated as eyedrops or eye ointments. These formulations may be prepared by conventional means, and, if desired, the compositions may be mixed with any conventional additive, such as an excipient, a binder, a disintegrating agent, a lubricant, a corrigent, a solubilizing agent, a suspension aid, an emulsifying agent or a coating agent.

In formulations of the subject invention, wetting agents, emulsifiers and lubricants, such as sodium lauryl sulfate and magnesium stearate, as well as coloring agents, release agents, coating agents, sweetening, flavoring and perfuming agents, preservatives and antioxidants may be present in the formulated agents.

Subject compositions may be suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of composition that may be combined with a carrier material to produce a single dose vary depending upon the subject being treated, the severity of hypertension, the medication status, and the particular mode of administration.

Methods of preparing these formulations include the step of bringing into association compositions of the present invention with the carrier and, optionally, one or more accessory ingredients. In general, the formulations are prepared by uniformly and intimately bringing into association agents with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition thereof as an active ingredient. Compositions of the present invention may also be administered as a bolus, electuary, or paste.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds or absorption descelerators, such as cristaline formulas; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent. Formulations which are suitable for vaginal administration also include pessaries, tampons, creams, gels, pastes, foams or spray formulations containing such carriers as are known in the art to be appropriate.

Dosage forms for transdermal administration of a subject composition includes powders, sprays, ointments, pastes, creams, lotions, gels, solutions, patches and inhalants. The active component may be mixed under sterile conditions with a pharmaceutically acceptable carrier, and with any preservatives, buffers, or propellants which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions and compounds of the present invention may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions.

Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions of this invention suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

In certain embodiments, the subject compounds may be formulated as a tablet, pill capsule or other appropriate ingestible formulation (collectively hereinafter “tablet”), to provide a therapeutic dose in 10 tablets or fewer. In another example, a therapeutic dose is provided in 50, 40, 30, 20, 15, 10, 5 or 3 tablets.

In a certain embodiment, the antihypertensive agent is formulated for oral administration as a tablet or an aqueous solution or suspension. In another embodiment of the tablet form of the antihypertensive agent, the tablets are formulated such that the amount of antihypertensive agent (or antihypertensive agents) provided in 20 tablets, if taken together, would provide a dose of at least the median effective dose (ED₅₀), e.g., the dose at which at least 50% of individuals exhibited the quantal effect of reduction in systolic and/or diastolic blood pressure. In a further embodiment, the tablets are formulated such that the total amount of antihypertensive agent (or antihypertensive agents) provided in 10, 5, 2 or 1 tablets would provide at least an ED₅₀ dose to a patient (human or non-human mammal). In other embodiments, the amount of antihypertensive agent (or antihypertensive agents) provided in 20, 10, 5 or 2 tablets taken in a 24 hour time period would provide a dosage regimen providing, on average, a mean plasma level of the antihypertensive agent(s) of at least the ED₅₀ concentration (the concentration for 50% of maximal effect of, e.g., reducing systolic and/or diastolic blood pressure). In other embodiments less than 100 times, 10 times, or 5 times the ED₅₀ is provided. In other embodiments, a single dose of tablets (1-20 tablets) provides about 0.25 mg to 1250 mg of an antihypertensive agent(s). In an exemplary embodiment, a single dose of tablets (1-20 tablets) provides about 0.5 to 5 mg, about 1 to about 3 mg, or about 2.5 mg of an antihypertensive agent.

Likewise, the antihypertensive agents can be formulated for parenteral administration, as for example, for subcutaneous, intramuscular or intravenous injection, e.g., the antihypertensive agent can be provided in a sterile solution or suspension (collectively hereinafter “injectable solution”). The injectable solution is formulated such that the amount of antihypertensive agent (or antihypertensive agents) provided in a 200 cc bolus injection would provide a dose of at least the median effective dose, or less than 100 times the ED₅₀, or less than 10 or 5 times the ED₅₀. The injectable solution may be formulated such that the total amount of antihypertensive agent (or antihypertensive agents) provided in 100, 50, 25, 10, 5, 2.5, or 1 cc injections would provide an ED₅₀ dose to a patient, or less than 100 times the ED₅₀, or less than 10 or 5 times the ED₅₀. In other embodiments, the amount of antihypertensive agent (or antihypertensive agents) provided in a total volume of 100 cc, 50, 25, 5 or 2 cc to be injected at least twice in a 24 hour time period would provide a dosage regimen providing, on average, a mean plasma level of the antihypertensive agent(s) of at least the ED₅₀ concentration, or less than 100 times the ED₅₀, or less than 10 or 5 times the ED₅₀. In other embodiments, a single dose injection provides about 0.25 mg to 1250 mg of an antihypertensive agent. In an exemplary embodiment, a single dose injection provides about 0.5 to 5 mg, about 1 to about 3 mg, or about 2.5 mg of an antihypertensive agent.

Dosages

Administration of the compositions of the present invention will be in an amount sufficient to achieve a therapeutic effect as recognized by one of ordinary skill in the art.

The dosage of any compositions of the present invention will vary depending on the symptoms, age and body weight of the patient, the nature and severity of the disorder to be treated or prevented, the route of administration, and the form of the subject composition. Any of the subject formulations may be administered in a single dose or in divided doses. Dosages for the compositions of the present invention may be readily determined by techniques known to those of skill in the art or as taught herein.

In certain embodiments, the dosage of the subject compounds will generally be in the range of about 0.01 ng to about 10 g per kg body weight, specifically in the range of about 1 ng to about 0.1 g per kg, and more specifically in the range of about 100 ng to about 10 mg per kg.

An effective dose or amount, and any possible affects on the timing of administration of the formulation, may need to be identified for any particular composition of the present invention. This may be accomplished by routine experiment as described herein, using one or more groups of animals (preferably at least 5 animals per group), or in human trials if appropriate. The effectiveness of any subject composition and method of treatment or prevention may be assessed by administering the composition and assessing the effect of the administration by measuring one or more applicable indices, and comparing the post-treatment values of these indices to the values of the same indices prior to treatment.

The precise time of administration and amount of any particular subject composition that will yield the most effective treatment in a given patient will depend upon the activity, pharmacokinetics, and bioavailability of a subject composition, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

While the subject is being treated, the health of the patient may be monitored by measuring one or more of the relevant indices at predetermined times during the treatment period. Treatment, including composition, a mounts, times of administration and formulation, may be optimized according to the results of such monitoring. The patient may be periodically reevaluated to determine the extent of improvement by measuring the same parameters. Adjustments to the amount(s) of subject composition administered and possibly to the time of administration may be made based on these reevaluations.

Treatment may be initiated with smaller dosages which are less than the optimum dose of the compound. Thereafter, the dosage may be increased by small increments until the optimum therapeutic effect is attained.

The use of the subject compositions may reduce the required dosage for any individual agent contained in the compositions (e.g., the steroidal anti inflammatory drug) because the onset and duration of effect of the different agents may be complimentary.

Toxicity and therapeutic efficacy of subject compositions may be determined by standard pharmaceutical procedures in cell cultures or experimental animals, e.g., for determining the LD₅₀ and the ED₅₀.

The data obtained from the cell culture assays and animal studies may be used in formulating a range of dosage for use in humans. The dosage of any subject composition lies preferably within a range of circulating concentrations that include the ED₅₀ with little or no toxicity. The dosage may vary within this range depending upon the dosage form employed and the route of administration utilized. For compositions of the present invention, the therapeutically effective dose may be estimated initially from cell culture assays.

In general, the doses of an active agent will be chosen by a physician based on the age, physical condition, weight and other factors known in the medical arts.

### Exemplification

The invention now being generally described, it will be more readily understood by reference to the following examples which are included merely for purposes of illustration of certain aspects and embodiments of the present invention, and are not intended to limit the invention in any way.

### EXAMPLE 1

### Daily Nighttime Administration of Melatonin Reduces Blood Pressure in Patients with Essential Hypertension

The endogenous circadian pacemaker, located in the suprachiasmatic nucleus (SCN), imposes 24-hour biological rhythms by endocrine and autonomic mechanisms (Buijs, R. M. Nat Rev Neurosci, 2 (2001) 521-6). For example, the circadian rhythm in adrenal cortex activity is regulated via both an endocrine and a sympathetic route, (Buijs, R. M., et al., European J. Neurosci, 11 (1999) 1535-1544; Scheer, F. A. J. L and Buijs, R. M., J. Clin. Endocrinol Metab., 84 (1999) 3395-3398) that of heart and liver via both sympathetic and parasympathetic control, (Fleur, S. E. et al., Brain Res., 871 (2000) 50-56; Scheer, F. A. J. L, et al., Am. J. Physiol. 280 (2001) H1391-H1399; Scheer. F. A. J. L, et al., J. Biol. Rhythms, 14 (1999) 202-212) and of the pineal gland via the sympathetic nervous system (Klein, D. C. and Weller, J. L, Science, 177 (1972) 532-533). Thus, the SCN promotes adaptation to the rest and activity periods by regulating—for example—the morning increase in cortisol, heart rate and glucose, and the evening increase in melatonin.

Evidence for disturbed circadian pacemaker function in essential hypertension is accumulating. Patients with hypertension show blunted day-night rhythms in sympathetic and parasympathetic heart tone (Guzzetti, S., et al., J. Hypertens., 9 (1991) 831-838; Nakano, Y, et al., Auton Neurosci, 88 (2001)181-6. Niijima, A., et al., Journal of the Autonomic Nervous System 40 (1992) 155-160). Patients with coronary heart disease—a major complication of chronic hypertension—show a blunted day-night rhythm in vasodilatation (Shaw, J. A., et al., Circulation, 103 (2001) 806-812) and suppressed nighttime melatonin levels (Brugger, P., et al., Lancet, 345 (1995) 1408). It has recently been demonstrated that, in comparison with normotensive subjects, the levels of three important SCN-neurotransmitters are reduced by more than 50% in patients with essential hypertension, (Goncharuk, V. D., et al., J. Comp. Neurology, 431 (2001) 320-330) corroborating its functional impairment. Furthermore, there is anatomical support for a changed SCN output to the sympathetic nervous system and to the hypothalamo-pituitary-adrenal axis in patients with essential hypertension (Goncharuk, V. D., et al., J Comp Neurol, 443 (2002) 321-31). Accordingly, compromised cardiovascular anticipation to the activity period in patients with essential hypertension may possibly lead to an increased risk of cardiovascular incidents in the early morning (Muller, J. E., et al., Circulation, 79 (1989) 733-43; Stergiou, G. S. et al., Stroke, 33 (2002) 1480-6).

Melatonin (N-acetyl-5-methoxy-tryptamine) secretion from the pineal gland is controlled by the SCN (Klein, D. C. and Weller, J. L, Science, 177 (1972) 532-533). Melatonin also provides feedback via high affinity melatonin receptors in the SCN, (Reppert, S. M., et al., Science, 242 (1988) 78-83; Weaver, D. R. and Reppert, S. M., NeuroReport, 8 (1996) 109-112) thus influencing the rhythm of its own production and other circadian rhythms (Bothorel, B.,et al., Eur J Neurosci, 16 (2002) 1-10; Zaidan, R, et al., Neuroendocrinology, 60 (1994) 105-12). Nighttime melatonin amplifies circadian rhythms directly via the central pacemaker, (Bothorel, B., et al., Eur J Neurosci, 16 (2002) 1-10; Zaidan, R., et al., Neuroendocrinology, 60 (1994) 105-12) and is used to improve disturbed day-night rhythms, (Arendt, J., et al., Ciba Found Symp, 117 (1985) 266-83) as in dementia, (Mishima, K., et al., Chronobiol. Int, 17 (2000) 419-432) shift work, (Sharkey, K. M. and Eastman, C. L, Am J Physiol Regul Integr Comp Physiol, 282 (2002) R454-63) and blindness (Sack. R. L., et al., N Engl J Med. 343 (2000) 1070-7). Because the SCN influences the autonomic output to the cardiovascular system, (Scheer, F. A. J. L, et al., Am. J. Physiol 280 (2001) H1391-H1399; Scheer. F. A. J. L, et al., J Biol. Rhythms, 14 (1999) 202-212; Sly, D. J., et al., Journal of the Autonomic Nervous System, 77 (1999) 73-82) restoration of proper functioning of the SCN in patients with hypertension could improve the autonomic regulation of blood pressure. A double blind, placebo-controlled crossover study, showing the effect of single and 3-week daily bedtime melatonin intake on ambulatory blood pressure in patients with essential hypertension is presented below.

Methods

Patients. Sixteen male patients with untreated, uncomplicated, essential hypertension were included in the study. Women were excluded to prevent possible interference of the menstrual cycle and oral anticonceptives on cardiovascular and circadian regulation. Patients were considered hypertensive, with a daytime ambulatory blood pressure between 140-179 mm Hg systolic or between 90-109 mm Hg diastolic. Secondary hypertension was excluded by medical history and routine laboratory tests. Five patients had never received antihypertensive medication; in the others antihypertensive treatment was stopped at least three weeks before participation. Their mean (±SD) age was 55±8 (range 36-68) years and mean BMI was 26.8±.1.7 (23.3-29.1) kg/m². Mean self reported habitual waking and bedtimes were 06:38 a.m. ±55 minutes and 11:29 p.m. ±26 minutes, respectively. The subjects did not travel across time zones or participate in shiftwork for at least 6 weeks before and during the study. Five days before and during ambulatory blood pressure measurements, subjects maintained fixed sleep-wake cycles according to their habitual sleep-wake cycle. All procedures were carried out with adequate understanding and written consent of the subjects and were approved by the Ethics Committee of the Academic Medical Center of the University of Amsterdam.

Study Design. The study had a balanced, randomized, double blind, placebo-controlled, crossover design. The effect of acute (1 day) and repeated (once daily for 3 weeks) melatonin on ambulatory blood pressure and heart rate was investigated. Melatonin (2.5 mg controlled-release (100% dissolved in 60 minutes); Terafarm, Katwijk, The Netherlands) or matching placebo was taken orally 1 hour before bedtime. On assessment days, patients abstained from heavy physical exercise, daytime napping, alcohol and nicotine consumption and coffee was restricted to two consumptions per day during breakfast or lunch. Meal times were restricted to the following periods: breakfast 1 to 1.5 hours after waking; lunch 11 to 10 hours before bedtime; and dinner 5 to 4 hours before bedtime.

Measurements. Four ambulatory blood pressure recordings were conducted (SpaceLabs 90207, Redmond, Wash., USA) (O'Brien. E., et al., BMJ, 322 (2001) 1110-4) while taking melatonin or placebo. During the first two recordings, 1 week apart, the acute placebo-controlled effect of melatonin was studied. During the last two recordings, 3 weeks apart, the repeated placebo-controlled melatonin effect was investigated. Ambulatory blood pressure was measured every half hour for 32 hours, from 8 hours before bedtime until bedtime the next day. In addition, noninvasive finger arterial blood pressure recordings were performed by Finometer (TNO Biomedical Instruments, Amsterdam, The Netherlands) (Bos, W. J., et al., Circulation, 94 (1996) 1870-5), to estimate changes in total peripheral resistance, stroke volume, and cardiac output, as possible explanations for changes in blood pressure. These recordings were performed before and at the end of both 3-week periods with melatonin or placebo. The three recordings were performed in the morning and at least 10 hours after melatonin or placebo intake in a quiet room during at least 10 minutes in supine position followed by at least 5 minutes standing. During all ambulatory blood pressure recordings, the subjects reported sleep details in a sleep-wake diary and wore an Actiwatch (Cambridge Neurotechnology Ltd, Cambridge, UK), a piezo-electric accelerometer, on the non-dominant wrist to assess motor activity at 1 min intervals for the estimation of sleep quality.

Data analysis. Sleep blood pressure was defined as the mean blood pressure from the time of falling asleep until the time of awakening, as determined by actigraphy. Awake blood pressure was defined as the mean blood pressure during the remaining portion of the day. Continuous Finometer data were analyzed by Beatscope software (TNO-Biomedical Instruments, Amsterdam, The Netherlands) (Bos, W. J., et al., Circulation, 94 (1996) 1870-5). Periods of 5 min with a stable blood pressure signal were used for both supine and standing periods. To accurately determine the sleeping period and estimate sleep quality, automatic sleep/wake scoring was performed with Actiwatch Sleep Analysis 98 (Cambridge Neurotechnology Ltd, V4.15), with sensitivity set to medium, on the actigraphy data between “bed time” and “get up time” derived from the sleep-wake diaries. Three sleep variables were objectively computed by this analysis. ‘Sleep latency’ was the calculated time between bed time and sleep onset. The time asleep was termed actual sleep time. The percentage of time asleep while in bed was defined as ‘sleep efficiency’.

Statistics. Because not all variables were normally distributed (Shapiro's-Wilk's W Test), non-parametric tests were used as required. Consequently, Altman's crossover analysis methods were applied (Altman, D. G., Crossover trials, Practical statistics for medical research, Vol. 1, Chapman and Hall. London, 1991, pp. 467-471). Paired student's t-tests or Wilcoxon matched pairs tests were applied to test differences between (1) single melatonin versus single placebo-period, and (2) repeated melatonin versus repeated placebo period. Student's t-tests for independent variables or Mann-Whitney U tests were applied for all dependent variables to test a carryover effect for (1) single treatment periods, and (2) repeated treatment periods. Correlation between a change in sleep quality and a change in sleep blood pressure was tested by linear correlation. The 24-hour rhythm in blood pressure was fitted by standard cyclic regression models: cosine, cosine with second harmonic, skewed cosine, peaked cosine, and skewed and peaked cosine analysis (Batschelet, E., Circular Statistics in Biology, Academic Press, 1981, 371 pp.). Of these, the latter best fitted the data as indicated by the Akaike's Information Criterion (AIC) (de Leeuw, J., introduction to Akaike (1973) Information Theory and an Extension of the Maximum Likelihood Principle. In S. Kotz and N. L. Johnson (Eds.), Breakthroughs in statistics, Vol. 1, Springer-Verlag. London, 1992 pp. 599-609) and was used for subsequent analysis. Two-tailed p values lower than 0.05 were considered to indicate statistical significance. All group data are presented as means±standard deviation (SD) or means including 95% confidence interval (95% CI).

Results

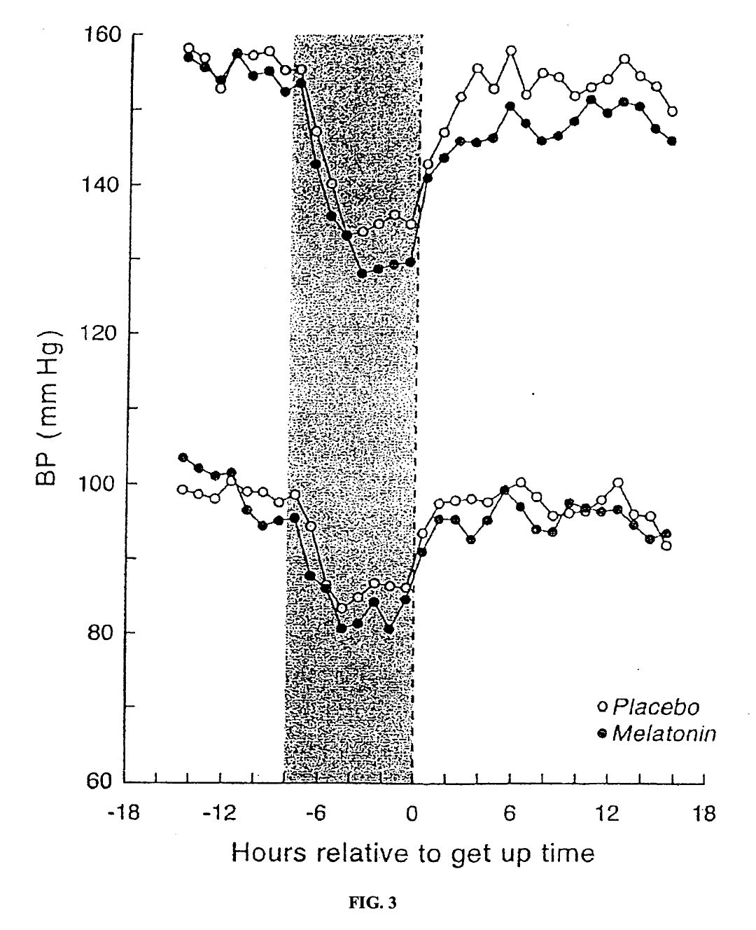

Ambulatory bloodpressure. Three weeks of 2.5 mg melatonin 1 hour before bedtime caused a significant reduction of sleep systolic and diastolic blood pressure of 6±10 (95% CI, −1 to −8) and 4±6 (−1 to −6) mm Hg as compared to 3 weeks placebo (p=0:046 and p=0.020), without a change in heart rate (p=0.23). There was no period effect (p=0.29 and p=0.76, for SBP and DBP) and no treatment-period interaction (p=0.18 and p=0.27, for SBP and DBP). Awake systolic and diastolic blood pressure did not decrease significantly when we compared 3 weeks melatonin with 3 weeks placebo (−5 and −1 mm Hg; p=0.14 and p=0.41). The blood pressure rhythms after repeated melatonin and placebo relative to get up time are shown in FIG. 3, illustrating the main effect at night and in the early morning hours. Acute melatonin application had no effect on systolic and diastolic blood pressure, whilst asleep (p=0.89 and p=0.86) or awake (p=0.20 and p=0.80).

TABLE 1
Average Blood Pressure and Heart Rate after 3 Weeks Placebo or Melatonin*.

| | Sleeping Period | | | Waking Period | | |
|---|---|---|---|---|---|---|
| | Systolic BP | Distolic BP | Heart Rate | Systolic BP | Distolic BP | Heart Rate |
| Placebo | 136.2 ± 14.1 | 86.3 ± 6.6 | 59.8 ± 6.6 | 152.8 ± 13.0 | 96.8 ± 7.8 | 69.6 ± 7.6 |
| Melatonin | 130.6 ± 10.0^{#} | 82.4 ± 4.0^{#} | 61.1 ± 6.5 | 147.6 ± 12.3 | 95.4 ± 7.7 | 71.2 ± 7.8 |

*Plus-minus values are means ± standard deviation. BP indicates blood pressure.
^{#}Significant difference compared to placebo treatment.

24-Hour rhythm analysis of ambulatory blood pressure. For 24-hour rhythm analysis of ambulatory blood pressure, the peaked and skewed cosine analysis was used. For patient 13 no significant fit was reached for systolic and diastolic blood pressure and for patient 3 not for systolic blood pressure, and these data were excluded from further analysis. Three-weeks melatonin enhanced the day-night rhythm amplitude of systolic and diastolic blood pressure by 15% (p=0.031) and 25% (p=0.029), respectively, as compared to 3 weeks placebo. The calculated minimum and mean diastolic blood pressure decreased by 5±7 and 4±6 mm Hg (p=0.008 and p=0.023), respectively, comparing 3 weeks melatonin with 3 weeks placebo. The decrease in minimum and mean systolic blood pressure was not significant (−6±15 mm Hg; p=0. 13 and −5±12 mm Hg; p=0. 19). There were no effects of repeated melatonin on maximum systolic and diastolic blood pressure (−2±12 mm Hg; p=0.50 and −1±5 mm Hg; p=0.49), respectively, or on time of the minimum blood pressure.

Finger arterial blood pressure recordings. There was no elect of repeated melatonin on blood pressure, heart rate, stroke volume, cardiac output, or total peripheral resistance during either supine resting conditions or standing during daytime as measured by Finometer.

Sleep-wake rhythm. Repeated melatonin significantly increased sleep efficiency (from 80% to 85%; p=0.017) and actual sleep time (from 5.6 hours to 6.1 hours; p=0.013) and significantly reduced sleep latency (from 33 to 22 minutes; p=0.036). There was no correlation between the effect of melatonin on any of the sleep variables and the effect of melatonin on sleep systolic and sleeping diastolic blood pressure. Acute melatonin application had no significant effect on sleep efficiency (p=0.39), actual sleep time (p=0. 18), or sleep latency (p=0.35).

Repeated but not single bedtime melatonin intake significantly reduced sleep blood pressure in male patients with untreated uncomplicated essential hypertension, by 6 and 4 mm Hg for systolic and diastolic blood pressure, respectively. A reduction of about 6 mm Hg over day and night, which reached significance during sleep, when blood pressure levels are most stable, is a meaningful reduction, since a drop of as little as 2-3 mm Hg systolic blood pressure has great clinical relevance (Staessen, J. A., et al., Lancet, 358 (2001) 1305-15). Furthermore, a reduction of sleep blood pressure by melatonin is important since we are asleep for approximately one third of our life and since nighttime blood pressure seems to better predict cardiovascular risk than daytime blood pressure (Staessen, J. A, et al., Systolic hypertension in Europe Trial Investigators, JAMA, 282 (1999) 539-46). Moreover, as FIG. 3 illustrates, bedtime melatonin might be beneficial in reducing the blood pressure also in the morning, a period when the blood pressure elevation may participate in the increased risk for cardiovascular incidents at that time (Muller, J. E., et al., Circulation, 79 (1989) 733-43; Stergiou, G. S. et al., Stroke, 33 (2002) 1480-6).

### Equivalents

The present disclosure provides among other things methods and compositions for treating hypertension. While specific embodiments have been discussed, the above specification is illustrative and not restrictive. Many variations of the methods, compositions, and process disclosed herein will become apparent to those skilled in the art upon review of this specification. The appended claims are not intended to claim all such embodiments and variations, and the full scope of the invention should be determined by reference to the claims, along with their full scope of equivalents, and the specification, along with such variations.

### Incorporation by Reference

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

Also incorporated by reference are the following: U.S. Pat. Nos. 6,110,951; 6,617,337; 5,232,945; 5,054,493; 5,804,204; 6,211,182; 6,589,572; 6,540,687; 4,803,218; 5,449,683; 4,665,086; U.S. patent application Publication No. 2002/0037887; Russian Patent No. RU2134108; Lusardi et al., Blood Pressure Monit. 2: 99-103; Cagnacci et al., Am J. Physiol. 274 (Regulatory Integrative Comp. Physiol. 43): R335-R338.; and Scheer, Cardiovascular Control by the Biological Clock. Neural and Neuroendocrine Mechanisms in Human and Rat. Ph.D. Thesis, University of Amsterdam, Amsterdam, The Netherlands (2003), ISBN: 90 6464 085 8; Scheer et al., J. Biol. Rhythms 14: 202-212 (1999); Scheer et al., Am. J. Physiol. 280: H1391-H1399 (2001); Scheer, et al., Biol. Chem. 384: 697-709 (2003); Tuma J, et al., Chronobiol Int. 18(5):781-99 (2001); Conway S, et al., Biochem Biophys Res Commun. 282(5):1229-36 (2001); Van Reeth O, et al., Am J Physiol Regul Integr Comp Physiol. 280(5):R1582-91 (2001); Weibel L, et al., Brain Res. 880(1-2):207-11 (2000); Ting N, et al., Naunyn Schmiedebergs Arch Pharmacol. 361(3):327-33 (2000); Loo H, et al., Encephale. 29(2):165-71 (2003); Millan M J, et al., Pharmacol Exp Ther. 306(3):954-64 (2003); Descamps-Francois C, et al., J Med Chem. 46(7):1127-9 (2003); Loo H, et al., Int Clin Psychopharmacol. 17(5):239-47 (2002); Nickelsen T, et al., Chronobiol Int. 19(5):915-36 (2002); Mohamed Naguib, et al., Anesth Analg 97:763-768 (2003); Vachharajani N N, et al., J Pharm Sci. 92(4):760-72 (2003); Mattson R J, et al., Bioorg Med Chem Lett. 13(6):1199-202 (2003); Patel R N, Curr Opin Biotechnol. 12(6):587-604 (2001); Uchikawa 0, et al., J Med Chem. 45(19):4222-39 (2002); Nickelsen et al., Chronobiol. Int. 19: 915-36 (200); and Vachharajani et al., J. Pharm. Sci. 92: 760-72 (2003).

1. A method for treating hypertension, comprising administering to a subject in need thereof a melatonin receptor agonist, wherein said melatonin receptor agonist is administered to said subject at night time or before the patient's bedtime on a daily basis, and wherein said melatonin receptor agonist reduces blood pressure in said subject thereby treating hypertension.

2. The method of claim 1, wherein the melatonin receptor agonist is administered at night time for at least 2 days.

3. The method of claim 2, wherein the melatonin receptor agonist is administered at night time for at least 7 days.

4. The method of claim 3, wherein the melatonin receptor agonist is administered at night time for at least 14 days.

5. The method of claim 4, wherein the melatonin receptor agonist is administered at night time for at least 21 days.

6. The method of claim 1, wherein the melatonin receptor agonist is a compound of Formula I: 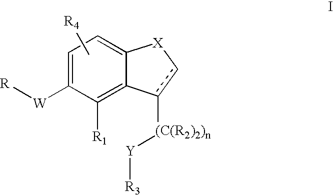
wherein, independently for each occurrence:
R is C₁₋₆ alkyl, C₂₋₈ alkenyl, C₂₋₈ alkynyl, cycloalkyl, aryl, aralkyl, heteroaryl, heteroaralkyl, —CO₂(C₁₋₆ alkyl), —CO₂(aryl), —C(O)NH(C₁₋₆ alkyl), —C(O)NH(aryl), —C(O)H, —C(O)(aryl), or —C(O)(C₁₋₆ alkyl);
R₁ is H, C₁₋₆ alkyl, C₂₋₈ alkenyl, C₂₋₈ alkynyl, aryl, aralkyl, heteroaryl, heteroaralkyl; or R and R₁ taken together form a fused aromatic, cycloalkyl, or cycloalkenyl ring;
R₂ is H, C₁₋₆ alkyl, cycloalkyl, aryl, aralkyl, or —CO₂R₁;
R₃ is H, C₁₋₆ alkyl, cycloalkyl, aryl, aralkyl, or —C(O)R₁;
R₄ is H, C₁₋₆ alkyl, or halide;
R₅ is H, C₁₋₆ alkyl, C₁₋₆ alkoxy; aryl, aralkyl, cycloalkyl, —N(R₁)₂, or heteroaryl;
W is O, N(R₂), or S;
X is CH₂, O, N(R₂) or S;
Y is O, N(R₆), or S;
R₆ is H, C₁₋₆ alkyl, —CO₂(C₁₋₆ alkyl); or R₆ and R₃ taken together form a ring;
the 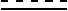
line indicates either a single or double bond between the two carbon atoms; and
n is an integer from 1 to 6 inclusive;
or a pharmaceutically acceptable salt, solvate, clathrate, polymorph, or co-crystal thereof.

7. The method of claim 1, wherein the melatonin receptor agonist is a compound of Formula II: 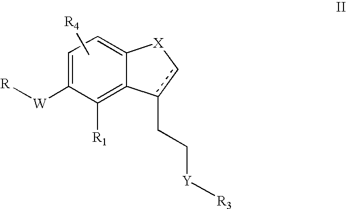
wherein, independently for each occurrence:
R is C₁₋₆ alkyl, C₂₋₈ alkenyl, C₂₋₈ alkynyl, cycloalkyl, aryl, aralkyl, heteroaryl, heteroaralkyl, —CO₂(C₁₋₆ alkyl), —CO₂(aryl), —C(O)NH(C₁₋₆ alkyl), —C(O)NH(aryl), —C(O)H, —C(O)(aryl), or —C(O)(C₁₋₆ alkyl);
R₁ is H, C₁₋₆ alkyl, C₂₋₈ alkenyl, C₂₋₈ alkynyl, aryl, aralkyl, heteroaryl, heteroaralkyl; or R and R₁ taken together form a fused aromatic, cycloalkyl, or cycloalkenyl ring;
R₃ is H, C₁₋₆ alkyl, cycloalkyl, aryl, aralkyl, or —C(O)R₁;
R₄ is H, C₁₋₆ alkyl, or halide;
W is O or S;
X is CH₂, O, N(R₁) or S;
Y is O, N(R₆), or S;
R₆ is H, C₁₋₆ alkyl, —CO₂(C₁₋₆ alkyl); or R₆ and R₃ taken together form a ring; and
the 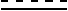
line indicates either a single or double bond between the two carbon atoms;
or a pharmaceutically acceptable salt, solvate, clathrate, polymorph, or co-crystal thereof.

8. The method of claim 1, wherein the melatonin receptor agonist is a compound of Formula III: 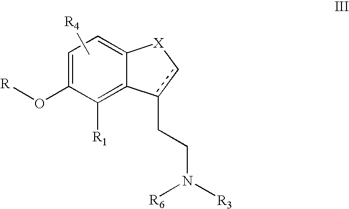
wherein, independently for each occurrence:
R is C₁₋₆ alkyl, C₂₋₈ alkenyl, C₂₋₈ alkynyl, cycloalkyl, aryl, aralkyl, heteroaryl, heteroaralkyl, —CO₂(C₁₋₆ alkyl), —CO₂(aryl), —C(O)NH(C₁₋₆ alkyl), —C(O)NH(aryl), —C(O)H, —C(O)(aryl), or —C(O)(C₁₋₆ alkyl);
R₁ is H, C₁₋₆ alkyl, C₂₋₈ alkenyl, C₂₋₈ alkynyl, aryl, aralkyl, heteroaryl, heteroaralkyl; or R and R₁ taken together form a fused aromatic, cycloalkyl, or cycloalkenyl ring;
R₃ is H, C₁₋₆ alkyl, cycloalkyl, aryl, aralkyl, or —C(O)R₁;
R₄ is H, C₁₋₆ alkyl, or halide;
X is CH₂ or N(R₁);
R₆ is H, C₁₋₆ alkyl, —CO₂(C₁₋₆ alkyl); or R₆ and R₃ taken together form a ring; and
the 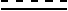
line indicates either a single or double bond between the two carbon atoms;
or a pharmaceutically acceptable salt, solvate, clathrate, polymorph, or co-crystal thereof.

9. The method of claim 1, wherein the melatonin receptor agonist is a compound of Formula IV: 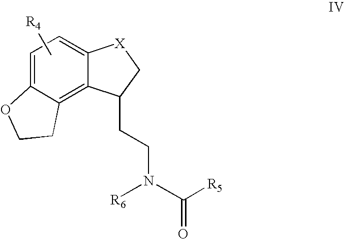
wherein, independently for each occurrence:
R₄ is H, C₁₋₆ alkyl, or halide;
R₅ is H, C₁₋₆ alkyl, C₁₋₆ alkoxy; aryl, aralkyl, cycloalkyl, or heteroaryl;
X is CH₂ or N(R₅); and
R₆ is H, C₁₋₆ alkyl, —CO₂(C₁₋₆ alkyl);
or a pharmaceutically acceptable salt, solvate, clathrate, polymorph, or co-crystal thereof.

10. The method of claim 1, wherein the melatonin receptor agonist is one or more of the following: N-[2-(5-Methoxy-1H-indol-3-yl)ethyl]acetamide (TAK-375), N-[2-(3-ethyl-7-methoxynaphthyl)ethyl]-acetamide (S21634), N-[2-(7-methoxynaphth-1-yl)-ethyl]-acetamide (S20098), N-[2-naphth-1-yl-ethyl]-cyclobutyl carboxamide (S20928), 2-iodomelatonin, N-acetyl-5-HT, LY 156735, BMS-214778, melatonin, agomelatine, CGP 52608, low-dose melatonin A, GR196429, S20242, S23478, S24268, S25150, melatonin receptor research compound A, GW290569, controlled release melatonin, luzindole, GR135531, melatonin agonist A, melatonin analogue B, melatonin agonist C, melatonin agonist D, melatonin agonist E, melatonin agonist F, melatonin agonist G, melatonin agonist H, melatonin agonist I, melatonin analog J , melatonin analog K, melatonin analog L, AH-001, GG-012, enol-3-IPA, ML-23, SL-18.1616, IP-100-9, melatonin low-dose B, sleep inducing peptide A, oros-melatonin, AH-0 17, AH-002, IP-101, 5-hydroxy-N-acetyl-tryptamine (NAT), 5-methoxy-N-bu-tanoyltryptamine (bMT), prazosin, phenylmelatonin, seradrene, β-methyl-6-chloromelatonin, 5-hydroxyethoxy-N-acetyltryptamine (5-HEAT), 8-methoxy-2-propionamidotetralin, PD-6735, seroctin, N-[2-(5-methoxy-2-phenylfuro[2,3-b]pyridin-3-yl)ethyl]acetamide, N-[2-(5-methoxy-2-phenylfuro[2,3-c]pyridin-3-yl)ethyl]acetamide, N-[(±)-2-(7-methoxy-1,2,3,4-tetrahydro-1-naphthyl)ethyl]cyclopropylcarboxamide, N-[2-(7-methoxy-1-naphthyl)ethyl]acetamide, N-acetyl-4-aminomethyl-6-methoxy-9-methyl-1,2,3,4-tetrahydrocarbazole (AMMTC), 3-(2-aminopropyl)indole, 6-chloromelatonin, 2,3-dihydromelatonin, 6-chloro-2,3-dihydromelatonin, N-acetyl-N′-formyl-5-methoxykynurenamine, 6-methoxybenzoxazolinone, or a pharmaceutically acceptable salt, solvate, clathrate, polymorph, or co-crystal thereof.

11. The method of claim 1, wherein the melatonin receptor agonist is one or more of the following: melatonin, N-[2-(5-Methoxy-1H-indol-3-yl)ethyl]acetamide (TAK-375), N-[2-(3-ethyl-7-methoxynaphthyl)ethyl]-acetamide (S21634), N-[2-(7-methoxynaphth-1-yl)-ethyl]-acetamide (S20098), N-[2-naphth-1-yl-ethyl]-cyclobutyl carboxamide (S20928), 2-iodomelatonin, N-acetyl-5-HT, LY 156735, BMS-214778, or a pharmaceutically acceptable salt, solvate, clathrate, polymorph, or co-crystal thereof.

12. The method of claim 1, wherein the melatonin receptor agonist is melatonin, or a pharmaceutically acceptable salt, solvate, clathrate, polymorph, or co-crystal thereof.

13. The method of claim 1, wherein the hypertension is essential hypertension.

14. The method of claim 1, wherein about 0.05, 0.1, 0.3, 1, 2.5, 5, 10, 20, 30, 50, 64, 100, 200, or 300 mg of said melatonin receptor agonist is administered to said subject at night time on a daily basis.

15. The method of claim 14, wherein about 2.5 mg of said melatonin receptor agonist is administered to said subject at night time on a daily basis.

16. The method of claim 1, wherein said melatonin receptor agonist is administered to said subject about 1 hour before the patient's bedtime.

17. The method of claim 1, wherein said melatonin receptor agonist is formulated for oral, nasal, parenteral or transdermal administration.

18. The method of claim 1, further comprising identifying a subject suffering from hypertension.

19. The method of claim 1, further comprising monitoring the blood pressure of said subject daily.

20. The method of claim 1, further comprising administering to said subject an anti-hypertensive agent that is not a melatonin receptor agonist.

21. A method for treating hypertension, comprising:
a) administering to a subject a melatonin receptor agonist at night time;
b) obtaining a blood pressure reading for said subject; and
c) repeating steps a) and b) on a daily basis until a desirable level of blood pressure reduction is achieved in said subject, thereby treating hypertension.

22. A program for treating hypertension in a subject, comprising:
a) determining a first blood pressure reading for a subject with hypertension;
b) providing a supply of a melatonin receptor agonist;
c) directing said subject to ingest a dose of said melatonin receptor agonist on a regular basis at night time for a period;
d) determining a second blood pressure reading for said subject at about the end of the period; and
e) comparing said second blood pressure reading with said first blood pressure reading to determine any reduction in hypertension in said subject during said period.

23. The method of claim 22, further comprising directing said subject to monitor said subject's blood pressure during said period.
